(19) United States
(12) Patent Application Publication
(10) Pub. No.: US 2020/0408746 A1
(43) Pub. Date: Dec. 31, 2020

(54) Stabilized Peptides for Biomarker Detection

(71) Applicant: Dana-Farber Cancer Institute, Inc., Boston, MA (US)

(72) Inventors: Loren D. Walensky, Newton, MA (US); Henry D. Herce, Brookline, MA (US); Rida Mourtada, Brookline, MA (US)

(21) Appl. No.: 16/968,644

(22) PCT Filed: Mar. 14, 2019
(86) PCT No.: PCT/US2019/022186

§ 371 Date: Aug. 10, 2020

Related U.S. Application Data
(60) Provisional application No. 62/643,018, filed on Mar. 14, 2018.

(51) Int. Cl. G01N 33/53; G01N 33/574

(52) U.S. Cl. CPC G01N 33/5306; G01N 2333/4703; G01N 33/57488

(57) ABSTRACT

This disclosure describes stabilized peptides and methods for detecting a biomarker on, outside, or within a live cell or tissue, or in a cell or tissue that has been fixed for an immunological assay or histopathologic evaluation. The present disclosure further describes stabilized peptides and methods for diagnosing and/or monitoring the progression of a disease in a subject, or for monitoring the efficacy of a disease treatment in a subject, based on the presence of a biomarker, the amount of a biomarker, and/or the localization of a biomarker in a live or fixed cell or tissue obtained from a subject.

### CROSS-REFERENCE TO RELATED APPLICATIONS

This application claims the priority benefit of U.S. provisional application No. 62/643,018, filed Mar. 14, 2018, which is hereby incorporated by reference herein in its entirety.

### BACKGROUND

As personalized medicine has emerged as a driving force in therapeutic decision-making, the detection of disease biomarkers has become critical for selecting appropriate therapies for particular patients. Various methodologies have been developed to interrogate patient samples for pathological mutations, proteins, and signaling networks that could be predictive of treatment outcomes. Immunohistopathology based on the use of antibodies that can bind to and be used to identify proteins associated with a disease in fixed tissue samples, such as biopsy specimens, remains the gold standard for disease diagnosis. However, reliable diagnostic antibodies have not been developed for many protein targets associated with numerous diseases, thereby hindering clinical testing and diagnosis for these diseases. Antibodies can be further limited to use in fixed cells and tissues, hindering the real-time analysis of the cellular dynamics of proteins implicated in disease. In addition, technical hurdles related to the widespread use of diagnostic antibodies, such as batch-to-batch variability in antibody quality, and relatively short antibody shelf-life, remain a challenge. As a result, new reagents are needed to detect and monitor disease-associated proteins in live and fixed cells and tissues.

### SUMMARY

The compositions and methods described herein enable the detection of one or more biomarkers, e.g., one or more polypeptides, proteins, and/or antigens, on, outside, or within a fixed or living cell or tissue using stabilized peptides, e.g., stapled and/or stitched peptides. In particular, the compositions and methods described herein represent a new approach to diagnostic testing by enabling the generation of stabilized peptides linked to detectable labels that can precisely and reliably bind to, and label, intracellular or extracellular proteins associated with disease, allowing for the histologic and pathologic identification of disease. Stabilized peptides have several advantages as diagnostic detection reagents compared to antibodies. The size and chemical properties of stabilized peptides allow for their use in detecting target polypeptides or proteins inside or outside living cells and tissues. Stabilized peptides also can be synthesized with high purity and low batch-to-batch variability, have a long shelf life, and display high affinity and specificity for their target proteins. Moreover, stabilized peptides can be designed to bind to protein pockets that are undisturbed by fixation solutions, permitting efficient detection of one or more biomarkers in fixed cells or tissues. Furthermore, stabilized peptides can be designed and produced to bind to protein targets for which there are no clinically useful antibodies available (e.g., certain BCL-2 family proteins).

In one aspect, the disclosure features a method for detecting a biomarker on, outside, or within a cell or tissue, the method comprising: (a) contacting a cell or tissue with a stabilized peptide that binds to the biomarker, wherein the stabilized peptide is linked to a detectable label; and (b) detecting the detectable label when the stabilized peptide is bound to the biomarker on, outside, or within the cell or tissue; thereby detecting the biomarker on, outside, or within the cell or tissue.

In some embodiments of this aspect, the stabilized peptide comprises a portion of a protein template (e.g., a wild-type or mutant polypeptide or protein, or portion thereof) that binds to the biomarker. In some embodiments, the stabilized peptide is 4 to 100 amino acids in length.

In some embodiments of this aspect, the stabilized peptide comprises at least one hydrocarbon staple or stitch. In some embodiments, the stabilized peptide comprises two hydrocarbon staples. In some embodiments, the hydrocarbon staples are separated by 2, 3, or 6 amino acids. In some embodiments, the hydrocarbon staples increase the stability and/or biomarker affinity of the stapled peptide on, outside, or within the cell relative to an identical peptide lacking the hydrocarbon staples.

In some embodiments of this aspect, the stabilized peptide further comprises at least one amino acid substitution relative to the protein template, e.g., wild-type or mutant polypeptide or protein, or portion thereof. In some embodiments, the at least one amino acid substitution increases the stability and/or biomarker affinity of the stabilized peptide on, outside, or within the cell or tissue relative to an identical stapled peptide lacking the at least one amino acid substitution.

In some embodiments of this aspect, the detectable label is a radioisotope, a fluorescent dye, an enzyme, or a substrate for an enzyme. In some embodiments, the detectable label is an affinity tag.

In some embodiments of this aspect, the cell is a live cell. In some embodiments, the tissue is a live tissue. In some embodiments, the cell has been fixed. In some embodiments, the tissue has been fixed. In some embodiments, the cell is a mammalian cell. In some embodiments, the cell is a non-mammalian cell.

In some embodiments of this aspect, the method further comprises the step of determining the level of detectable label on, outside, or within the cell or tissue relative to a control cell or tissue. In some embodiments, the method further comprises determining the intracellular location of the detectable label in the cell.

In another aspect, the disclosure features a method for detecting a biomarker on, outside, or within a live cell, the method comprising: (a) providing a peptide that binds to the biomarker; (b) introducing at least one staple and/or stitch into the peptide to produce a stabilized peptide that binds to the biomarker; (c) contacting the cell or tissue with the stabilized peptide that binds to the biomarker; and (d) detecting the stabilized peptide when the stabilized peptide is bound to the biomarker, thereby detecting the biomarker on, outside, or within the cell.

In some embodiments of this aspect, the stabilized peptide is linked to a detectable label. In some embodiments, the detectable label is a radioisotope, a fluorescent dye, an enzyme, or a substrate for an enzyme. In some embodiments, the detectable label is an affinity tag.

In some embodiments of this aspect, the stabilized peptide comprises a portion of a protein template (e.g., a wild-type or mutant polypeptide or protein, or portion thereof) that binds to the biomarker. In some embodiments, the stabilized peptide is 4 to 100 amino acids in length.

In some embodiments of this aspect, the stabilized peptide comprises at least one hydrocarbon staple. In some embodiments, the stabilized peptide comprises two hydrocarbon staples. In some embodiments, the hydrocarbon staples are separated by 2, 3, or 6 amino acids. In some embodiments, the hydrocarbon staples increase the stability and/or biomarker affinity of the stabilized peptide on, outside, or within the cell relative to an identical peptide lacking the hydrocarbon staples.

In some embodiments, the stapled peptide comprises or consists of an amino acid sequence set forth in any one of SEQ ID Nos.: 2 to 44.

In some embodiments of this aspect, the stabilized peptide further comprises at least one amino acid substitution relative to the protein template, e.g., wild-type or mutant polypeptide or protein, or portion thereof. In some embodiments, the at least one amino acid substitution increases the stability and/or biomarker affinity of the stabilized peptide on, outside, or within the cell or tissue relative to an identical stapled peptide lacking the at least one amino acid substitution.

In some embodiments, the cell is a mammalian cell. In some embodiments, the cell is a non-mammalian cell.

In some embodiments of this aspect, the method further comprises the step of determining the level of detectable label on, outside, or within the cell or tissue relative to a control cell or tissue. In some embodiments, the method further comprises determining the intracellular location of the detectable label in the cell.

In another aspect, the disclosure features a kit for detecting a biomarker on, outside, or within a cell or tissue, the kit comprising: (a) a stabilized peptide that binds to the biomarker, wherein the stabilized peptide is linked to a detectable label; and (b) instructions for using the stabilized peptide to detect the biomarker on, outside, or within the cell or tissue.

In some embodiments of this aspect, the kit further comprises reagents for detecting the detectable label on, outside, or within the cell or tissue.

In some embodiments of this aspect, the stabilized peptide comprises a portion of a protein template (e.g., a wild-type or mutant polypeptide or protein, or portion thereof) that binds to the biomarker. In some embodiments, the stabilized peptide is 4 to 100 amino acids in length.

In some embodiments of this aspect, the stabilized peptide comprises at least one hydrocarbon staple. In some embodiments, the stabilized peptide comprises two hydrocarbon staples. In some embodiments, the hydrocarbon staples are separated by 2, 3, or 6 amino acids. In some embodiments, the hydrocarbon staples increase the stability and/or biomarker affinity of the stabilized peptide on, outside, or within the cell relative to an identical peptide lacking the hydrocarbon staples.

In some embodiments, the stapled peptide comprises or consists of an amino acid sequence set forth in any one of SEQ ID Nos.: 2 to 44.

In some embodiments of this aspect, the stabilized peptide further comprises at least one amino acid substitution relative to the protein template, e.g., wild-type or mutant polypeptide or protein, or portion thereof. In some embodiments, the at least one amino acid substitution increases the stability and/or biomarker affinity of the stabilized peptide on, outside, or within the cell or tissue relative to an identical stapled peptide lacking the at least one amino acid substitution.

In some embodiments, the detectable label is a radioisotope, a fluorescent dye, an enzyme, or a substrate for an enzyme. In some embodiments, the detectable label is an affinity tag.

In some embodiments of this aspect, the kit comprises instructions for detecting the biomarker on, outside, or within a live cell or tissue.

In some embodiments of this aspect, the kit comprises instructions for detecting the biomarker on, outside, or within a fixed cell or tissue.

In some embodiments of this aspect, the cell is a live cell. In some embodiments, the tissue is a live tissue. In some embodiments, the cell has been fixed. In some embodiments, the tissue has been fixed. In some embodiments, the cell is a mammalian cell. In some embodiments, the cell is a non-mammalian cell.

In some embodiments of this aspect, the kit further comprises instructions and reagents for determining the level of detectable label on, outside, or within the cell or tissue relative to a control cell or tissue. In some embodiments, the kit further comprises instructions and reagents for determining the intracellular location of the detectable label in the cell or tissue.

In another aspect, the disclosure features a method for selecting a stabilized peptide that binds to a biomarker, the method comprising: providing a peptide that binds to the biomarker; introducing at least one staple and/or stitch into the peptide to produce a first stabilized peptide that binds to the biomarker; contacting the cell or tissue with the first stabilized peptide linked to a detectable label; detecting that the first stabilized peptide binds to the biomarker better and/or for a longer period of time relative to the peptide and/or a second stabilized peptide generated from the peptide, and selecting the first stabilized peptide for detecting the biomarker.

In some embodiments of this aspect, the detectable label is a fluorescent dye. In some embodiments, the detectable label is a radioisotope, an enzyme, or a substrate for an enzyme. In some embodiments, the detectable label is an affinity tag.

In some embodiments of this aspect, the stabilized peptide comprises a portion of a protein template (e.g., a wild-type or mutant polypeptide or protein, or portion thereof) that binds to the biomarker. In some embodiments, the stabilized peptide is 4 to 100 amino acids in length.

In some embodiments of this aspect, the stabilized peptide comprises at least one hydrocarbon staple. In some embodiments, the stabilized peptide comprises two hydrocarbon staples. In some embodiments, the hydrocarbon staples are separated by 2, 3, or 6 amino acids. In some embodiments, the hydrocarbon staples increase the stability and/or biomarker affinity of the stabilized peptide on, outside, or within the cell relative to an identical peptide lacking the hydrocarbon staples.

In some embodiments of this aspect, the stabilized peptide further comprises at least one amino acid substitution relative to the protein template, e.g., wild-type or mutant polypeptide or protein, or portion thereof. In some embodiments, the at least one amino acid substitution increases the stability and/or biomarker affinity of the stabilized peptide on, outside, or within the cell or tissue relative to an identical stapled peptide lacking the at least one amino acid substitution.

In some embodiments of this aspect, the biomarker is a BCL-2 family protein. In some embodiments, the biomarker is selected from the group consisting of BFL-1, MCL-1, β-catenin, β-amyloid, tau, α-synuclein, TDP-43, Polycomb protein EED, HDM2, HDMX, WT1, MUC1, LMP2, HPV E6, HPV E7, EGFRvIII, HER-2/neu, MAGE A3, p53, NY-ESO-1, PSMA, GD2, CEA, MelanA/MART1, Ras, gp100, Proteinase3 (PR1), bcr-abl, Tyrosinase, Survivin, PSA, hTERT, Sarcoma translocation breakpoints, EphA2, PAP, ML-IAP, AFP, EpCAM, ERG, NA17, PAX3, ALK, Androgen receptor, Cyclin B1, Polysialic acid, MYCN, RhoC, TRP-2, GD3, Fucosyl GM1, Mesothelin, PSCA, MAGE A1, sLe, CYP1B1, PLAC1, GM3, BORIS, Tn, GloboH, ETV6-AML, NY-BR-1, RGSS, SART3, STn, Carbonic anhydrase IX, PAXS, OY-TES1, Sperm protein 17, LCK, HMWMAA, AKAP-4, SSX2, XAGE 1, 7H3, Legumain, Tie 2, Page4, VEGFR2, MAD-CT-1, FAP, PDGFR-β, MAD-CT-2, Fos-related antigen 1, PCNA, GLP1 receptor, RAS proteins, glucokinase, VLCAD, RSV, or HIV.

In some embodiments, the stapled peptide comprises or consists of an amino acid sequence set forth in any one of SEQ ID Nos.: 2 to 44.

In another aspect, the disclosure features a method of detecting a biomarker in a solution, the method comprising: (a) providing a solution comprising a biomarker; (b) contacting the solution with a stabilized peptide that binds to the biomarker, wherein the stabilized peptide is linked to a detectable label; (c) detecting the detectable label when the stabilized peptide is bound to the biomarker in the solution; thereby detecting the biomarker in the solution.

In some embodiments of this aspect, the solution is blood, serum, plasma, urine, mucous, cerebrospinal fluid, a lavage, pleural fluid, vaginal fluid, semen, peritoneal fluid, or a secretion. In certain embodiments, the secretion is sweat or saliva. In certain embodimetns, the solution is blood.

In some embodiments of this aspect, the stabilized peptide is linked to a solid carrier. In some embodiments, the solid carrier is a magnetic bead.

In some embodiments of this aspect, the stabilized peptide binds to the biomarker in solution better and/or for a longer period of time relative to the peptide from which the stabilized peptide is derived or relative to a second stabilized peptide also derived from the same peptide.

In some embodiments, the stapled peptide comprises or consists of an amino acid sequence set forth in any one of SEQ ID Nos.: 2 to 44.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 depicts the amino acid sequences of stapled polypeptides derived from the N-terminus of the S. cerevisiae Abp140 protein (peptide (a) (SEQ ID NO:1)), including the location of hydrocarbon staples (X) in peptides (b)-(h) and an alanine substitution in peptides (g) and (h). The 17-amino acid sequences of (b)-(h) are assigned SEQ ID NOs.: 2-7 and 44, respectively. “B” is norleucine. “KT” is Lysine(TAMRA), i.e., a lysine residue with the fluorescent dye 5-carboxytetramethylrhodamine (TAMRA) attached to its side chain amine. “KBio” is Lysine(Biotin), i.e., a lysine residue with a biotin attached to its side chain amine. “X” is a stapling amino acid, such as (S)-2-(4-pentenyl)alanine.

FIG. 2A depicts a confocal image showing actin filaments labeled with stapled peptide (g) (SEQ ID NO: 7) in living cells.

FIG. 2B depicts a confocal image of Hoechst DNA dye labeling of the nuclei of the cells in FIG. 2A.

FIG. 2C depicts a transmitted light (TL) image of the cells in FIG. 2A.

FIG. 2D depicts an overlay of the three imaging channels of FIGS. 2A-C.

FIG. 3A depicts a confocal image showing actin filaments labeled with stapled peptide (g) (SEQ ID NO: 7) in fixed cells.

FIG. 3B depicts a confocal image of Hoechst DNA dye labeling of the nuclei of the cells in FIG. 3A.

FIG. 3C depicts labeling of the actin filament network by FITC-phalloidin (Actistain 488).

FIG. 3D depicts an overlay of the three imaging channels of FIGS. 3A-C.

FIG. 4A shows a fluorescently-tagged stapled actin-binding peptide (SEQ ID NO:7) labeling actin in U2OS cells.

FIG. 4B demonstrates an alternative method for detecting the stapled peptide marker for actin using a biotinylated stapled peptide (SEQ ID NO:44) that can be visualized by applying fluorescently-tagged streptavidin to fixed cells, highlighting the versatility of the biomarker detection approach.

FIG. 5 shows that an exemplary stapled actin-binding peptide (SEQ ID NO:7) can label actin in fixed murine tissues, such as heart, lower extremity, and tongue muscle.

FIG. 6A demonstrates the intracellular distribution of expressed GFP-BFL-1/A1, highlighting the endogenous localization at the mitochondria.

FIG. 6B and FIG. 6C show that a selective BFL-1/A1 targeting stapled peptide marker, biotinylated-NOXA SAHB-15 (SEQ ID NO:17), colocalizes with GFP-BFL-1/A1 at the mitochondria in fixed cells.

FIG. 7A depicts how co-transfection of GFP-BFL-1/A1 with the chimeric protein GBP-LaminB1 can target BFL-1/A1 to the nuclear lamina, a non-endogenous localization for the protein.

FIG. 7B and FIG. 7C show how the selective BFL-1/A1 targeting stapled peptide now illuminates the nuclear lamina in fixed cells, highlighting the capacity of this stapled peptide marker to specifically detect its relocalized protein target.

FIG. 8A depicts how co-transfection of GFP-HDM2 with the chimeric protein GBP-LaminB1 can target HDM2 to the nuclear lamina in fixed cells, a non-endogenous localization for the protein.

FIG. 8B and FIG. 8C show how the HDM2 targeting stapled peptide, ATSP-7041 (SEQ ID NO:26) illuminates the nuclear lamina in fixed cells, highlighting the capacity of this stapled peptide marker to specifically detect its relocalized protein target.

FIG. 9 shows that an exemplary stapled actin-binding peptide (SEQ ID NO:7) can label actin in the skin of live zebrafish.

FIG. 10 top panel shows the chemical structures of exemplary unnatural amino acids used to generate various kinds of staples.

FIG. 10 middle panel illustrates peptides with staples of various lengths.

FIG. 10 bottom panel illustrates a staple walk along a peptide sequence.

FIG. 11 is a schematic showing representations of various kinds of double and triple stapling strategies along with exemplary staple walks.

FIG. 12 is a schematic showing exemplary staple walks using various lengths of branched double staple moieties.

FIG. 13 is a schematic showing exemplary chemical alterations that are employed to generate stapled peptide derivatives.

### DETAILED DESCRIPTION

Immunohistochemistry is a widely used diagnostic tool for identifying the presence of specific proteins in tissue samples, and determining protein levels in the samples. However, this diagnostic tool relies on the development of reliable antibodies that can bind their target proteins efficiently, typically within a fixed sample. Fixation solutions introduce cross links between amino acids, which has the potential of destroying the binding of an antibody to its antigen (see, e.g., Otali et al., Biotech Histochem, 84(5):223-247, 2009). Since antibodies can be difficult to develop and manufacture, especially antibodies that retain binding to their target antigens in a fixed cell or tissue environment, there are many important disease targets for which diagnostic antibodies have not been developed. Stabilized peptide reagents (e.g., stapled and/or stitched peptides) can precisely detect protein targets in both fixed and live cells, and thus represent an entirely new platform for tissue diagnostics.

Stabilized peptide reagents are particularly useful in fixed cells, as they can be designed to bind the target protein at sites that are undisturbed by the fixation process. In particular, because the binding site on the stabilized peptide's target protein is mostly hydrophobic, the binding site is less affected by fixation solutions. As a result, the binding site for the stabilized peptide reagent is sufficiently preserved, even after fixation, to enable the “helix-in-groove” interaction between the binding site and a stabilized helical peptide reagent.

Moreover, in contrast to antibody production, which generally requires expensive animal facilities and high production costs, e.g., to generate and maintain hybridomas for monoclonal antibody production, stabilized peptides can be reliably, reproducibly, and cost-effectively synthesized at large scales to target a broad diversity of proteins of interest. In addition, whereas immunohistochemical detection requires fixed tissue and a complicated and multistep processing protocol, stabilized peptides can be used to detect proteins in fixed or live cells and tissues with less processing, and even in a single step.

Although peptides are attractive candidates for the development of detection reagents for biomarkers, such as proteins implicated in disease, technical challenges have hindered their adoption as diagnostic reagents. For example, when a peptide sequence is removed from its native protein environment, it often loses its secondary structure, which is critical for its specific engagement of a target. The stabilized peptides and methods described herein solve this problem by introducing chemical constraints into the peptide sequences to reinforce the biological-recognition fold. By tailoring such stabilized peptides with fluorescent or affinity tags for diagnostic use, the stabilized peptides described herein are powerful and diverse detection reagents for proteins in fixed or living cells and tissues.

### Stabilized Peptides

A peptide helix is an important mediator of key protein-protein interactions that regulate many important biological processes; however, when such a helix is taken out of its context within a protein and prepared in isolation, it usually adopts a random coil conformation, leading to a drastic reduction in biological activity, such as ability to bind target proteins. To avoid this problem, one can employ structurally stabilized peptides. In some cases, structurally stabilized peptides comprise at least two modified amino acids joined by an internal (intramolecular) cross-link (or staple). Stabilized peptides as described herein include stapled peptides, stitched peptides, peptides containing multiple stitches, peptides containing multiple staples, or peptides containing a mix of staples and stitches, as well as peptides structurally reinforced by other chemical strategies (see, e.g., Balaram P. Cur. Opin. Struct. Biol. 1992;2:845; Kemp D S, et al., J. Am. Chem. Soc. 1996;118:4240; Orner B P, et al., J. Am. Chem. Soc. 2001;123:5382; Chin J W, et al., Int. Ed. 2001;40:3806; Chapman R N, et al., J. Am. Chem. Soc. 2004;126:12252; Horne W S, et al., Chem., Int. Ed. 2008;47:2853; Madden et al., Chem Commun (Camb). 2009 Oct 7; (37): 5588-5590; Lau et al., Chem. Soc. Rev., 2015,44:91-102; and Gunnoo et al., Org. Biomol. Chem., 2016,14:8002-8013; all of which are incorporated by reference herein in their entirety).

In certain embodiments, polypeptides can be stabilized by peptide stapling (see, e.g., Walensky, J. Med. Chem., 57:6275-6288 (2014), the contents of which are incorporated by reference herein in its entirety). A peptide is “stabilized” in that it maintains its native secondary structure. For example, stapling allows a polypeptide, predisposed to have an a-helical secondary structure, to maintain its native a-helical conformation. This secondary structure increases resistance of the polypeptide to proteolytic cleavage and heat, and also may increase target binding affinity, hydrophobicity, and cell permeability. Accordingly, the stapled (cross-linked) polypeptides described herein have improved biological activity relative to a corresponding non-stapled (un-cross-linked) polypeptide.

“Peptide stapling” is a term coined from a synthetic methodology wherein two olefin-containing side-chains (e.g., cross-linkable side chains) present in a polypeptide chain are covalently joined (e.g., “stapled together”) using a ring-closing metathesis (RCM) reaction to form a cross-linked ring (see, e.g., Blackwell et al., J. Org. Chem., 66: 5291-5302, 2001; Angew et al., Chem. Int. Ed. 37:3281, 1994). As used herein, the term “peptide stapling” includes the joining of two (e.g., at least one pair of) double bond-containing side-chains, triple bond-containing side-chains, or double bond-containing and triple bond-containing side chain, which may be present in a polypeptide chain, using any number of reaction conditions and/or catalysts to facilitate such a reaction, to provide a singly “stapled” polypeptide. The term “multiply stapled” polypeptides refers to those polypeptides containing more than one individual staple, and may contain two, three, or more independent staples of various spacing. Additionally, the term “peptide stitching,” as used herein, refers to multiple and tandem “stapling” events in a single polypeptide chain to provide a “stitched” (e.g., tandem or multiply stapled) polypeptide, in which two staples, for example, are linked to a common residue. Peptide stitching is disclosed, e.g., in WO 2008/121767 and WO 2010/068684, which are both hereby incorporated by reference in their entirety. In some instances, staples, as used herein, can retain the unsaturated bond or can be reduced.

In certain embodiments, polypeptides can be stabilized by, e.g., hydrocarbon stapling. In certain instances, the stapled peptide includes at least two (e.g., 2, 3, 4, 5, 6) amino acid substitutions, wherein the substituted amino acids are separated by two, three, or six amino acids, and wherein the substituted amino acids are non-natural amino acids with olefinic side chains. There are many known non-natural or unnatural amino acids any of which may be included in the stapled peptides. Some examples of unnatural amino acids are 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and /para-substituted phenylalanines (e.g., substituted with —C(═O)C₆H₅; —CF₃; —CN; -halo; —NO₂; CH₃), disubstituted phenylalanines, substituted tyrosines (e.g., further substituted with —C═O)C₆H₅; —CF₃; —CN; -halo; —NO₂; CH₃), and statine. Additionally, amino acids can be derivatized to include amino acid residues that are hydroxylated, phosphorylated, sulfonated, acylated, or glycosylated.

Hydrocarbon stapled polypeptides include one or more tethers (linkages) between two non-natural amino acids, which tether significantly enhances the a-helical secondary structure of the polypeptide. Generally, the tether extends across the length of one or two helical turns (i.e., about 3.4 or about 7 amino acids). Accordingly, amino acids positioned at i and i+3; i and i+4; or i and i+7 are ideal candidates for chemical modification and cross-linking. Thus, for example, where a peptide has the sequence . . . X1, X2, X3, X4, X5, X6, X7, X8, X9 . . . , cross-links between X1 and X4, or between X1 and X5, or between X1 and X8 are useful hydrocarbon stapled forms of that peptide, as are cross-links between X2 and X5, or between X2 and X6, or between X2 and X9, etc. The use of multiple cross-links (e.g., 2, 3, 4, or more) is also contemplated. The use of multiple cross-links is very effective at stabilizing and optimizing the peptide, especially with increasing peptide length. Thus, the disclosure encompasses the incorporation of more than one cross-link within the polypeptide sequence to either further stabilize the sequence or facilitate the structural stabilization, proteolytic resistance, acid stability, thermal stability, cellular permeability, and/or biological activity enhancement of longer polypeptide stretches. Additional description regarding making and use of hydrocarbon stapled polypeptides can be found, e.g., in U.S. Patent Publication Nos. 2012/0172285, 2010/0286057, and 2005/0250680, the contents of all of which are incorporated by reference herein in their entireties.

In certain embodiments when a staple is at the i and i+3 residues, R-propenylalanine and S-pentenylalanine; or R-pentenylalanine and S-pentenylalanine are substituted for the amino acids at those positions. In certain embodiments when a staple is at the i and i+4 residues, S-pentenyl alanine is substituted for the amino acids at those positions. In certain embodiments when a staple is at the i and i+7 residues, an S-pentenyl alanine and R-octenyl alanine, or R-pentenyl alanine and S-octenyl alanine, pair are substituted for the amino acids at those positions. In some instances, when the peptide is stitched, the amino acids of the peptide to be involved in the “stitch” are substituted with Bis-pentenylglycine, S-pentenylalanine, and R-octenylalanine; or Bis-pentenylglycine, S-octenylalanine, and R-octenylalanine.

Staple or stitch positions can be varied by testing different staple locations in a staple walk.

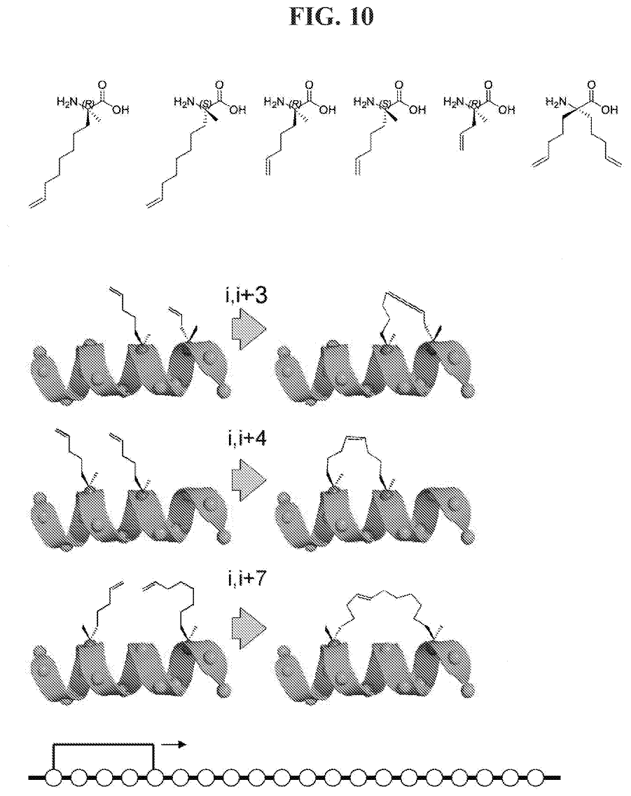

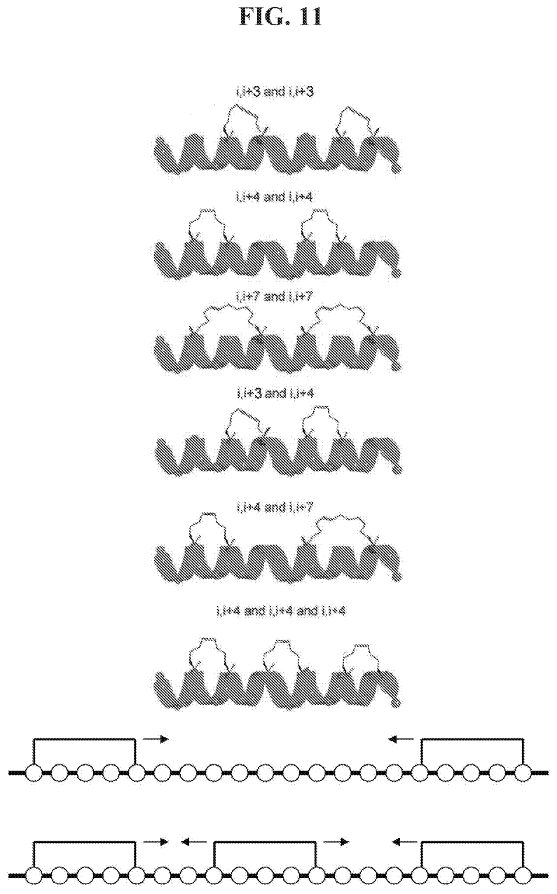

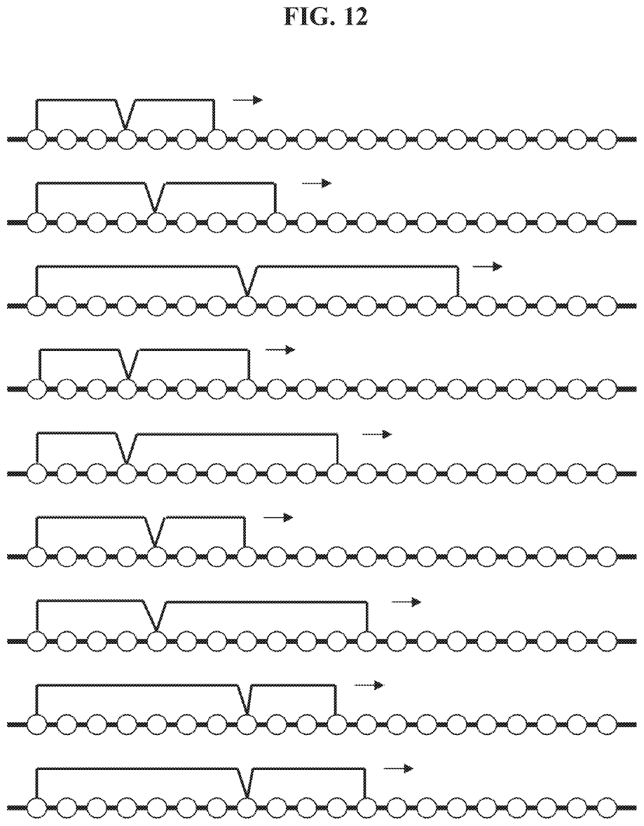

FIG. 10 shows exemplary chemical structures of non-natural amino acids that can be used to generate various cross-linked compounds. FIG. 10 also illustrates peptides with hydrocarbon cross-links between positions i and i+3; i and i+4; and i and i+7 residues. FIG. 10 also illustrates a staple walk along a peptide sequence. FIG. 11 shows various peptide sequences with double and triple stapling strategies, and exemplary staple walks. FIG. 12 illustrates exemplary staple walks using various lengths of branched stitched moieties.

In one aspect, a stabilized polypeptide has the formula (I),

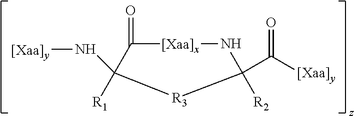

wherein:

each R₁ and R₂ are independently H or a C₁ to C₁₀ alkyl, alkenyl, alkynyl, arylalkyl, cycloalkylalkyl, heteroarylalkyl, or heterocyclylalkyl;
R₃ is alkyl, alkenyl, alkynyl; [R₄—K—R_{4]}ₙ; each of which is substituted with 0-6 R₅;
R₄ is alkyl, alkenyl, or alkynyl;
R₅ is halo, alkyl, OR₆, N(R₆)₂, SR₆, SOR₆, SO₂R₆, CO₂R₆, R₆, a fluorescent moiety, or
a radioisotope;

K is O, S, SO, SO₂, CO, CO₂, CONR₆, or

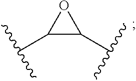 [SMILES: *C1OC1*]

R₆ is H, alkyl, or a therapeutic agent;

n is an integer from 1-4;
x is an integer from 2-10;
each y is independently an integer from 0-100;
z is an integer from 1-10 (e.g., 1, 2, 3, 4, 5, 6, 7, 8, 9, 10);

and each Xaa is independently an amino acid.

The tether can include an alkyl, alkenyl, or alkynyl moiety (e.g., C₅, C₈, or C₁₁ alkyl, a C₅, C₈, or C₁₁ alkenyl, or C₅, C₈, or C₁₁ alkynyl). The tethered amino acid can be alpha disubstituted (e.g., C₁-C₃ or methyl).

In some instances, x is 2, 3, or 6. In some instances, each y is independently an integer between 1 and 15, or 3 and 15. In some instances, R₁ and R₂ are each independently H or C₁-C₆ alkyl. In some instances, R₁ and R₂ are each independently C₁-C₃ alkyl. In some instances, at least one of R₁ and R₂ are methyl. For example, R₁ and R₂ can both be methyl. In some instances, R₃ is alkyl (e.g., C₈ alkyl) and x is 3. In some instances, R₃ is C₁₁ alkyl and x is 6. In some instances, R₃ is alkenyl (e.g., C₈ alkenyl) and x is 3. In some instances, x is 6 and R₃ is C₁₁ alkenyl. In some instances, R₃ is a straight chain alkyl, alkenyl, or alkynyl. In some instances, R₃ is —CH₂—CH₂—CH₂—CH═CH—CH₂—CH₂—CH₂—.

In another aspect, the two alpha, alpha disubstituted stereocenters are both in the R configuration or S configuration (e.g., i, i+4 cross-link), or one stereocenter is R and the other is S (e.g., i, i+7 cross-link). Thus, where formula I is depicted as:

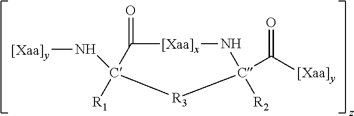

the C′ and C″ disubstituted stereocenters can both be in the R configuration or they can both be in the S configuration, e.g., when x is 3. When x is 6, the C′ disubstituted stereocenter is in the R configuration and the C″ disubstituted stereocenter is in the S configuration. The R₃ double bond can be in the E or Z stereochemical configuration.

In some instances, R₃ is [R₄—K—R₄]ₙ; and R₄ is a straight chain alkyl, alkenyl, or alkynyl.

In some embodiments, the disclosure features internally cross-linked (“stapled” or “stitched”) peptides, wherein the side chains of two amino acids separated by two, three, or six amino acids are replaced by an internal staple; the side chains of three amino acids are replaced by an internal stitch; the side chains of four amino acids are replaced by two internal staples, or the side chains of five amino acids are replaced by the combination of an internal staple and an internal stitch. The stapled/stitched peptide can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

In certain embodiments, the stabilized peptide is produced by introducing one or more staples or stitches into a protein or polypeptide template. The protein or polypeptide template can bind to a biomarker of interest, e.g., a polypeptide, protein, or antigen. In some embodiments, the protein or polypeptide template is a protein or polypeptide, or portion thereof, having a wild-type sequence. In some embodiments, the protein or polypeptide template is a protein or polypeptide, or portion thereof, having one or more mutations relative to the wild-type sequence of the protein or polypeptide, or portion thereof

In certain instances, the stabilized peptide is a peptide of an intracellular protein, or a portion thereof. In certain instances, the stabilized peptide is a peptide of an extracellular protein, or a portion thereof. In certain instances, the stabilized peptide is a peptide of protein with an extracellular component or a portion thereof In certain instances, the stabilized peptide is a peptide of a secreted protein or a portion thereof. In certain instances, the stabilized peptide is a peptide of a protein used in intracellular or intercellular signaling, or a portion thereof. In certain instances, the stabilized peptide is a peptide of a receptor, e.g., an extracellular or intracellular receptor, or a portion thereof. In certain instances, the stabilized peptide is a peptide of a receptor ligand, or a portion thereof. In certain instances, the stabilized peptide is a peptide of a disease causing or disease-related protein, or a portion thereof. In certain instances, the stabilized peptide is a peptide of a bacterial protein, or a portion thereof. In certain instances, the stabilized peptide is a peptide of a viral protein, or a portion thereof (e.g., HIV gp120). In certain instances, the stabilized peptide is a peptide of a protein of a pathogen, or a portion thereof. In certain instances, the stabilized peptide is a peptide of a protein of a plant, or a portion thereof. In certain instances, the stabilized peptide is a peptide of a protein of a fungi, or a portion thereof. In certain instances, the stabilized peptide is a peptide of a protein of an archaebacteria, or a portion thereof. In certain instances, the stabilized peptide is a peptide of a human protein, or a portion thereof. In certain instances, the stabilized peptide is a peptide of an oncogenic protein, or a portion thereof. In certain embodiments, the stabilized peptide is a peptide of a protein involved in neurologic disease, or a portion thereof. In certain embodiments, the stabilized peptide is a peptide of an animal protein, or a portion thereof.

A stabilized peptide described herein can be designed to bind to any target biomarker of interest, e.g., a protein implicated in a disease or cellular process. In certain instances, the stabilized peptide binds to at least one biomarker. The term “biomarker” refers to a protein, polypeptide, or other macromolecule, or portion thereof, that would be desirable to recognize or label. The skilled artisan will understand that any macromolecule, including almost all proteins, polypeptides, peptides or nucleic acids, can serve as an biomarker. Furthermore, biomarkers can be derived from nucleic acids, e.g., recombinant or genomic DNA. Any DNA, which comprises a nucleotide sequence or a partial nucleotide sequence encoding a protein that is desirable to recognize (whether by a reagent or the immune system), encodes an “biomarker” as that term is used herein. Moreover, a biomarker need not be encoded by a “gene” at all, but can be generated or synthesized or can be derived from a biological sample, or might be a macromolecule besides a polypeptide. Such a biological sample can include, but is not limited to, a tissue sample (e.g., tissue from a healthy subject or tissue from a diseased subject, such as a tumor sample), a cell or a fluid. In some embodiments, a biomarker can be an antigen and capable of eliciting an immune response. In other embodiments, a biomarker can be non-antigenic.

In certain instances, the stabilized peptide binds specifically to a single biomarker, e.g., binds a single biomarker with a high affinity. In certain instances, the biomarker is from a pathogen. In certain instances, the biomarker is from a bacterium, e.g., a bacterial protein. In certain instances, the biomarker is from a virus, e.g., a viral protein. In some instances, the stabilized peptide binds to an animal biomarker, e.g., a mammalian or avian protein. In some instances, the stabilized peptide binds to a human biomarker, e.g., a human protein. In some instances, the stabilized peptide binds to a plant biomarker, e.g., a plant protein. In some instances, the stabilized peptide binds to an biomarker on a cell, e.g., on the surface of the cell. For example, stabilized peptides described herein can bind to proteins having an extracellular component, such as, e.g., transmembrane proteins, including proteins involved in extracellular signaling such as transmembrane receptors or integrins (e.g., αvβ5, αvβ6, αvβ1). In some instances, the stabilized peptides bind to an biomarker outside of a cell, e.g., outside of a mammalian cell. For example, stabilized peptides described herein can bind to the extracellular matrix or components of the extracellular matrix. In some instances, stabilized peptides described herein can bind to extracellular proteins produced by bacteria or fungi, such as flagella or other polymeric fibers. In some instances, the stabilized peptides bind to a biomarker inside of a cell, e.g., a mammalian cell.

In certain instances, the biomarker is protein that can be used to predict the presence or absence of disease in a subject, e.g., a mammalian subject such as a human. In some instances, or relative likelihood of disease based on whether the protein is detected or not detected. In certain instances, the biomarker is protein that can be used to predict the presence, absence or relative likelihood of disease based on whether the protein is detectable or not detectable in a sample. In some embodiments, the biomarker is a protein that can be used to predict the relative likelihood of disease based on the levels of the protein in a sample relative to the levels in a control sample.

In some instances, the stabilized peptide can bind and detect an oncogenic protein, i.e., a protein whose presence, absence, amount, expression level, and/or localization can be used to detect or diagnose cancer, e.g., from a cancer biopsy. Non-limiting examples of oncogenic proteins include BCL2, BCLXL, MCL-1, BFL-1, BCL-w, BCL-B, EZH2, HDM2/HDMX, KRAS/NRAS/HRAS, MYC, β-catenin, PI3K, PTEN, TSC, AKT, BRCA1/2, a EWS-FLI fusion, an MLL fusion, a receptor tyrosine kinase, a HOX homolog, JUN, Cyclin D, Cyclin E, BRAF, CRAF, CDK4, CDK2, HPV-E6/E7, Aurora kinase, MITF, Wnt1, PD-1, BCR, and CCR5.

In some embodiments, a stabilized peptide described herein, e.g., a stapled and/or stitched peptide, can bind to and be used to detect any of the following biomarkers: β-catenin, EED, HDM2, HDMX, β-amyloid, tau, a-synuclein, TDP-43, HIV gp120, WT1, MUC1, LMP2, HPV E6 E7, EGFRvIII, HER-2/neu, Idiotype, MAGE A3, p53 wild type and nonmutants, NY-ESO-1, PSMA, GD2, CEA, MelanA/MART1, Ras wild type and mutants, gp100, Proteinase3 (PR1), bcr-abl, Tyrosinase, Survivin, PSA, hTERT, Sarcoma translocation breakpoints, EphA2, PAP, ML-IAP, AFP, EpCAM, ERG, NA17, PAX3, ALK, Androgen receptor, Cyclin B1, Polysialic acid, MYCN, RhoC, TRP-2, GD3, Fucosyl GM1, Mesothelin, PSCA, MAGE A1, sLe, CYP1B1, PLAC1, GM3, BORIS, Tn, GloboH, ETV6-AML, NY-BR-1, RGSS, SART3, STn, Carbonic anhydrase IX, PAXS, OY-TES1, Sperm protein 17, LCK, HMWMAA, AKAP-4, SSX2, XAGE 1, 7H3, Legumain, Tie 2, Page4, VEGFR2, MAD-CT-1, FAP, PDGFR-β, MAD-CT-2, Fos-related antigen 1, PCNA, GLP1 receptor, RAS proteins, glucokinase, VLCAD, RSV, or HIV.

Non-limiting examples of stabilized peptides that can be employed in the methods described herein are provided in U.S. Pat. Nos. 9,074,009; 8,637,260; 8,921,323; 9,822,165; 9,505,816; 9,079,970; U.S. Patent Application Publication Nos. 20170247423;; 20170015716; 20140018302; 20140370042; 2016/0031959; 2016/0046671; 2016/0068834; 2010/0273704; and WO 2017040329, WO 2017040323, WO 2017147283, WO 2017165617, WO 2018006009, and PCT/US2017048582, the contents of all of which are incorporated by reference in their entirety herein. In some embodiments, the stabilized peptide further comprises a detectable label.

Some non-limiting examples of stabilized peptides that can be employed in the methods described herein are listed in Table 1:

TABLE 1
Examples of stabilized peptides

| | Sequence | |
|---|---|---|
| Peptide | (SEQ ID NO) | Target |
| BIM SAHB_{A1} | IWIAQELRXIGDXFNAYYARR | BCL-2 family proteins |
| | (SEQ ID NO: 8) | |
| D-NA-BIM SAHB | *IAQELRXIGDXFNAYYARR | BFL-1, BCL-2 family |
| | (SEQ ID NO: 9) | proteins |
| BID SAHB | DIIRNIARHLAXVGDXBDRSI | BCL-2 family proteins |
| | (SEQ ID NO: 10) | |
| PUMA SAHB_{A2} | QWAREIGAQLRXBADXLNAQYERR | BCL-2 family proteins |
| | (SEQ ID NO: 11) | |
| BAD SAHB | NLWAAQRYGRELRXBSDXFVDSFKK | BCL-2, BCL-XL, BCL-w |
| | (SEQ ID NO: 12) | |
| BAD SAHB S155pS | NLWAAQRYGRELRXBpSDXFVDSFKK | Glucokinase |
| | (SEQ ID NO: 13) | |
| BAD SAHB S155D | NLWAAQRYGRELRXBDDXFVDSFKK | Glucokinase |
| | (SEQ ID NO: 14) | |
| NOXA SAHB (21-43) | AELEVECATQLRXFGDXLNFRQKLL | MCL-1, BFL-1 |
| | (SEQ ID NO: 15) | |
| D-NA-NOXA SAHB (22-43) | *EVESATQLRXFGDXLNFRQKLL | MCL-1, BFL-1 |
| | (SEQ ID NO: 16) | |
| D-NA-NOXA SAHB (26-40) | *AT8LRRFGDXLNFRQ | BFL-1 |
| | (SEQ ID NO: 17) | |
| D-NA-NOXA SAHB R31E | *AT8LREFGDXLNFRQ | BFL-1 |
| | (SEQ ID NO: 18) | |
| D-NA-NOXA SAHB F32A | *AT8LRRAGDXLNFRQ | BFL-1 |
| | (SEQ ID NO: 19) | |
| MCL-1 SAHB_{D} | RKALETLRRVGDGVXRNHXTAF | MCL-1 |
| | (SEQ ID NO: 20) | |
| MCL-1 SAHB_{D} V220A | RKALETLRRVGDGAXRNHXTAF | VLCAD |
| | (SEQ ID NO: 21) | |
| MCL-1 SAHB_{D} D218A | RKALETLRRVGAGVXRNHXTAF | VLCAD |
| | (SEQ ID NO: 22) | |
| SAH-MS1-14 | IWBXQSLXRLGDEINAYYARR | MCL-1 |
| | (SEQ ID NO: 23) | |
| SAH-MS1-18 | IWBXQELXRLGDEINARYAR | MCL-1 |
| | (SEQ ID NO: 24) | |
| SAH-p53-8 | QSQQTFXNLWRLLXQN | HDMX/HDM2 |
| | (SEQ ID NO: 25) | |
| ATSP-7041 | LTFZEYWAQOXSAA | HDMX/HDM2 |
| | (SEQ ID NO: 26) | |
| SAH-EZH2_{A} (40-68) | SNLFSSNRXKILXRTEILNQEWKQRRIQPV | EED |
| | (SEQ ID NO: 27) | |
| SAH-EZH2_{A} (42-68) | FSSNRXKILXRTEILNQEWKQRRIQPV | EED |
| | (SEQ ID NO: 28) | |
| SAH-EZH2_{B} (40-68) | SNLFSSNRQKILERTXILNXEWKQRRIQPV | EED |
| | (SEQ ID NO: 29) | |
| SAH-EZH2_{B} (42-68) | FSSNRQKILERTXILNXEWKQRRIQPV | EED |
| | (SEQ ID NO: 30) | |
| SAH-EZH2_{AB} (40-68) | SNLFSSNRXKILXRTXILNXEWKQRRIQPV | EED |
| | (SEQ ID NO: 31) | |
| SAH-EZH2_{AB} (42-68) | FSSNRXKILXRTXILNXEWKQRRIQPV | EED |
| | (SEQ ID NO: 32) | |
| SAH-BCL9_{B} | LSQEQLEHRERSLXTLRXIQRBLF | beta-catenin |
| | (SEQ ID NO: 33) | |
| SAH-RSVF_{BF} | FDZSISQVNXKINQSLAFIRKZDELLHNXNAG | RSV |
| | KST | |
| | (SEQ ID NO: 34) | |
| SAH-GP41_{AB} (626-662) | BTWXEWDXEINNYTSLIHSLIEESQNQXEKN | HIV |
| | XQELLE | |
| | (SEQ ID NO: 35) | |
| SAH-Ex_{AB} | HGEGTFTSDXSKQXEEEAVRLFIXWLKXGGP | GLP1 receptor |
| | SSGAPPPS | |
| | (SEQ ID NO: 36) | |
| SAH-BCL-2 BH4 | EIVBKYIHYKLSXRGYXWDA | IP3R, BAX |
| | (SEQ ID NO: 37) | |
| SAH-BCL-XL BH4 | RELVVDFLSYKLSXKGYXWSQ | IP3R, BAX |
| | (SEQ ID NO: 38) | |
| SAH-BCL-W BH4 | ALVADFVGYKLRXKGYXBGA | IP3R, BAX |
| | (SEQ ID NO: 39) | |
| SAH-SOS1_{A} | RRFFGIXLTNXLKTEEGN | RAS |
| | (SEQ ID NO: 40) | |
| NOXA SAHBA | LEVESATQLRXFGDXLNFRQKL | MCL-1, BFL-1 |
| | (SEQ ID NO: 41) | |
| EZH2 | FSSNRXKILXRTQILNQEWKQRRIQPV | EED |
| | (SEQ ID NO: 42) | |
| HRK | QLTAARLKXLGDXLHQRTBWR | |
| | (SEQ ID NO: 43) | |

wherein Z = R-octenyl alanine; B = norleucine; O = cyclobutylalanine; * = (R)-1 acryloylpiperidine-3-carboxamide; X = (S)-2- (4′-pentenyl) alanine; X₁ and X₂ = a non-natural amino acid, or other residue that permits stapling, and, in some instances, X₁ and X₂ are the same (e.g., (S)-2- (4′-pentenyl) alanine).

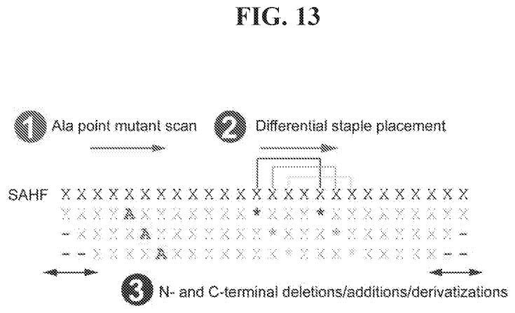

In certain embodiments, this disclosure features stabilized peptides that differ from the peptides disclosed above in that they vary in the location of the staple/stitch. In certain embodiments, this disclosure features stabilized peptides that differ from the peptides disclosed above in that they vary from the above-disclosed sequences in having 1 to 7 (e.g., 1, 2, 3, 4, 5, 6, 7) amino acid substitutions on the non-interacting face of the alpha-helix of these peptides. In certain instances, the substitutions are conservative. In other instances, the substitutions are non-conservative. In certain embodiments, this disclosure features stabilized peptides that differ from the peptides disclosed above in that they vary from the above-disclosed sequences in having 1 to 5 (e.g., 1, 2, 3, 4, 5) amino acid substitutions on the interacting face of the alpha-helix of these peptides. In certain instances, the substitutions are conservative. Exemplary types of variations/modifications to stapled peptides are illustrated in FIG. 13.

While hydrocarbon tethers are common, other tethers can also be employed in the stabilized peptides described herein. For example, the tether can include one or more of an ether, thioether, ester, amine, or amide, or triazole moiety. In some cases, a naturally occurring amino acid side chain can be incorporated into the tether. For example, a tether can be coupled with a functional group such as the hydroxyl in serine, the thiol in cysteine, the primary amine in lysine, the acid in aspartate or glutamate, or the amide in asparagine or glutamine. Accordingly, it is possible to create a tether using naturally occurring amino acids rather than using a tether that is made by coupling two non-naturally occurring amino acids. It is also possible to use a single non-naturally occurring amino acid together with a naturally occurring amino acid. Triazole-containing (e.g., 1,4 triazole or 1,5 triazole) crosslinks can be used (see, e.g., Kawamoto et al. 2012 Journal of Medicinal Chemistry 55:1137; WO 2010/060112). In addition, other methods of performing different types of stapling are well known in the art and can be employed (see, e.g., Lactam stapling: Shepherd et al., J. Am. Chem. Soc., 127:2974-2983 (2005); UV-cycloaddition stapling: Madden et al., Bioorg. Med. Chem. Lett., 21:1472-1475 (2011); Disulfide stapling: Jackson et al., Am. Chem. Soc.,113:9391-9392 (1991); Oxime stapling: Haney et al., Chem. Commun., 47:10915-10917 (2011); Thioether stapling: Brunel and Dawson, Chem. Commun., 552-2554 (2005); Photoswitchable stapling: J. R. Kumita et al., Proc. Natl. Acad. Sci. U S. A., 97:3803-3808 (2000); Double-click stapling: Lau et al., Chem. Sci., 5:1804-1809 (2014); Bis-lactam stapling: J. C. Phelan et al., J. Am. Chem. Soc., 119:455-460 (1997); and Bis-acylation stapling: A. M. Spokoyny et al., J. Am. Chem. Soc., 135:5946-5949 (2013)).

It is further envisioned that the length of the tether can be varied. For instance, a shorter length of tether can be used where it is desirable to provide a relatively high degree of constraint on the secondary alpha-helical structure, whereas, in some instances, it is desirable to provide less constraint on the secondary alpha-helical structure, and thus a longer tether may be desired.

Additionally, while tethers spanning from amino acids i to i+3, i to 1+4, and i to i+7 are common in order to provide a tether that is primarily on a single face of the alpha helix, the tethers can be synthesized to span any combinations of numbers of amino acids and also used in combination to install multiple tethers.

In some instances, the hydrocarbon tethers (i.e., cross links) described herein can be further manipulated. In one instance, a double bond of a hydrocarbon alkenyl tether, (e.g., as synthesized using a ruthenium-catalyzed ring closing metathesis (RCM)) can be oxidized (e.g., via epoxidation, aminohydroxylation or dihydroxylation) to provide one of compounds below.

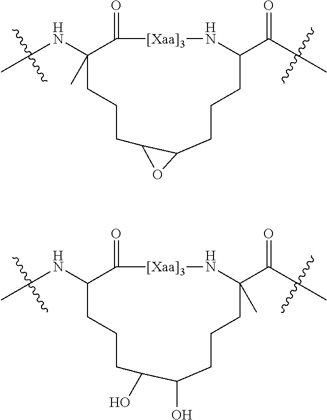

Either the epoxide moiety or one of the free hydroxyl moieties can be further functionalized. For example, the epoxide can be treated with a nucleophile, which provides additional functionality that can be used, for example, to attach a therapeutic agent. Such derivatization can alternatively be achieved by synthetic manipulation of the amino or carboxy-terminus of the polypeptide or via the amino acid side chain. Other agents can be attached to the functionalized tether, e.g., an agent that facilitates entry of the polypeptide into cells.

In some instances, alpha disubstituted amino acids are used in the polypeptide to improve the stability of the alpha helical secondary structure. However, alpha disubstituted amino acids are not required, and instances using mono-alpha substituents (e.g., in the tethered amino acids) are also envisioned. The addition of polyethylene glycol (PEG) molecules can improve the pharmacokinetic and pharmacodynamic properties of the polypeptide. For example, PEGylation can reduce renal clearance and can result in a more stable plasma concentration. PEG is a water soluble polymer and can be represented as linked to the polypeptide as formula:

XO═(CH₂CH₂O)n═CH₂CH₂═Y where n is 2 to 10,000 and X is H or a terminal modification, e.g., a C₁₋₄ alkyl; and Y is an amide, carbamate or urea linkage to an amine group (including but not limited to, the epsilon amine of lysine or the N-terminus) of the polypeptide. Y may also be a maleimide linkage to a thiol group (including but not limited to, the thiol group of cysteine). Other methods for linking PEG to a polypeptide, directly or indirectly, are known to those of ordinary skill in the art. The PEG can be linear or branched. Various forms of PEG including various functionalized derivatives are commercially available.

PEG having degradable linkages in the backbone can be used. For example, PEG can be prepared with ester linkages that are subject to hydrolysis. Conjugates having degradable PEG linkages are described in WO 99/34833; WO 99/14259, and U.S. Pat. No. 6,348,558.

In certain embodiments, macromolecular polymer (e.g., PEG) is attached to an agent described herein through an intermediate linker. In certain embodiments, the linker is made up of from 1 to 20 amino acids linked by peptide bonds, wherein the amino acids are selected from the 20 naturally occurring amino acids. Some of these amino acids may be glycosylated, as is well understood by those in the art. In other embodiments, the 1 to 20 amino acids are selected from glycine, alanine, proline, asparagine, glutamine, and lysine. In other embodiments, a linker is made up of a majority of amino acids that are sterically unhindered, such as glycine and alanine. Non-peptide linkers are also possible. For example, alkyl linkers such as —NH(CH₂)ₙC(O)—, wherein n=2-20 can be used. These alkyl linkers may further be substituted by any non-sterically hindering group such as lower alkyl (e.g., C₁-C₆) lower acyl, halogen (e.g., Cl, Br), CN, NH₂, phenyl, etc. U.S. Pat. No. 5,446,090 describes a bifunctional PEG linker and its use in forming conjugates having a peptide at each of the PEG linker termini.

The stabilized peptides can also be modified, e.g., to further facilitate cellular uptake or increase in vivo stability, in some embodiments. For example, acylating or PEGylating a peptidomimetic macrocycle facilitates cellular uptake, increases bioavailability, increases blood circulation, alters pharmacokinetics, decreases immunogenicity and/or decreases the needed frequency of administration.

In some embodiments, the stapled peptides disclosed herein have an enhanced ability to penetrate cell membranes (e.g., relative to non-stapled peptides).

In some embodiments, the stapled peptides are derivatized with a warhead (e.g. acrylamide or analog thereof) to covalently crosslink to the target biomarker, e.g., target protein or polypeptide.

Methods of synthesizing the stabilized peptides described herein are known in the art. Nevertheless, the following exemplary method may be used. It will be appreciated that the various steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 3d. Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The stabilized peptides can be made by chemical synthesis methods, which are well known to the ordinarily skilled artisan. See, for example, Fields et al., Chapter 3 in Synthetic Peptides: A User's Guide, ed. Grant, W. H. Freeman & Co., New York, N.Y., 1992, p. 77. Hence, peptides can be synthesized using the automated Merrifield techniques of solid phase synthesis with the α-NH₂ protected by either t-Boc or Fmoc chemistry using side chain protected amino acids on, for example, an Applied Biosystems Peptide Synthesizer Model 430A or 431.

One manner of making of the peptides described herein is using solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Any side chain functional groups are protected with base stable, acid labile groups.

Longer peptides could be made by conjoining individual synthetic peptides using native chemical ligation. Alternatively, the longer synthetic peptides can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

The peptides can be made in a high-throughput, combinatorial fashion, e.g., using a high-throughput multiple channel combinatorial synthesizer available from Advanced Chemtech.

Peptide bonds can be replaced, e.g., to increase physiological stability of the peptide, by: a retro-inverso bonds (C(O)—NH); a reduced amide bond (NH—CH₂); a thiomethylene bond (S—CH₂ or CH₂—S); an oxomethylene bond (O—CH₂ or CH₂—O); an ethylene bond (CH₂—CH₂); a thioamide bond (C(S)—NH); a trans-olefin bond (CH═CH); a fluoro substituted trans-olefin bond (CF═CH); a ketomethylene bond (C(O)—CHR) or CHR—C(O) wherein R is H or CH₃; and a fluoro-ketomethylene bond (C(O)—CFR or CFR—C(O) wherein R is H or F or CH₃.

The polypeptides can be further modified by: acetylation, amidation, biotinylation, cinnamoylation, farnesylation, fluoresceination, formylation, myristoylation, palmitoylation, phosphorylation (Ser, Tyr or Thr), stearoylation, succinylation and sulfurylation. As indicated above, peptides can be conjugated to, for example, polyethylene glycol (PEG); alkyl groups (e.g., C1-C20 straight or branched alkyl groups); fatty acid radicals; and combinations thereof

α, α-Disubstituted non-natural amino acids containing olefinic side chains of varying length can be synthesized by known methods (Williams et al. J. Am. Chem. Soc., 113:9276, 1991; Schafmeister et al., J. Am. Chem Soc., 122:5891, 2000; and Bird et al., Methods Enzymol., 446:369, 2008; Bird et al., Current Protocols in Chemical Biology, 2011). For peptides where an i linked to i+7 staple is used (two turns of the helix stabilized) either: a) one S5 amino acid and one R8 is used; or b) one S8 amino acid and one R5 amino acid is used. R8 is synthesized using the same route, except that the starting chiral auxiliary confers the R-alkyl-stereoisomer. Also, 8-iodooctene is used in place of 5-iodopentene. Inhibitors are synthesized on a solid support using solid-phase peptide synthesis (SPPS) on MBHA resin (see, e.g., WO 2010/148335).

Fmoc-protected α-amino acids (other than the olefinic amino acids Fmoc-S₅—OH, Fmoc-R₈—OH, Fmoc-S₈—OH and Fmoc-R₅—OH), 2-(6-chloro-1-H-benzotriazole-1-yl)-1,1,3,3-tetramethylaminium hexafluorophosphate (HCTU), and Rink Amide MBHA are commercially available from, e.g., Novabiochem (San Diego, Calif.). Dimethylformamide (DMF), N-methyl-2-pyrrolidinone (NMP), N,N-diisopropylethylamine (DIEA), trifluoroacetic acid (TFA), 1,2-dichloroethane (DCE), fluorescein isothiocyanate (FITC), and piperidine are commercially available from, e.g., Sigma-Aldrich. Olefinic amino acid synthesis is reported in the art (Williams et al., Org. Synth., 80:31, 2003).

Again, methods suitable for obtaining (e.g., synthesizing), stapling, and purifying the peptides disclosed herein are also known in the art (see, e.g., Bird et. al., Methods in Enzymol., 446:369-386 (2008); Bird et al., Current Protocols in Chemical Biology, 2011; Walensky et al., Science, 305:1466-1470 (2004); Schafmeister et al., J. Am. Chem. Soc., 122:5891-5892 (2000); U.S. patent application Ser. No. 12/525,123, filed Mar. 18, 2010; and U.S. Pat. No. 7,723,468, issued May 25, 2010, each of which are hereby incorporated by reference in their entirety).

In some embodiments, the peptides are substantially free of non-stapled peptide contaminants or are isolated. Methods for purifying peptides include, for example, synthesizing the peptide on a solid-phase support. Following cyclization, the solid-phase support may be isolated and suspended in a solution of a solvent such as DMSO, DMSO/dichloromethane mixture, or DMSO/NMP mixture. The DMSO/dichloromethane or DMSO/NMP mixture may comprise about 30%, 40%, 50% or 60% DMSO. In a specific embodiment, a 50%/50% DMSO/NMP solution is used. The solution may be incubated for a period of 1, 6, 12 or 24 hours, following which the resin may be washed, for example with dichloromethane or NMP. In one embodiment, the resin is washed with NMP. Shaking and bubbling an inert gas into the solution may be performed.

Properties of the stabilized (e.g., stapled) polypeptides of the invention can be assayed, for example, using the methods described below.

Assays to Determine α-Helicity: Compounds are dissolved in an aqueous solution (e.g. 5 mM potassium phosphate solution at pH 7, or distilled H₂O, to concentrations of 25-50 μM). Circular dichroism (CD) spectra are obtained on a spectropolarimeter (e.g., Jasco J-710, Aviv) using standard measurement parameters (e.g. temperature, 20° C.; wavelength, 190-260 nm; step resolution, 0.5 nm; speed, 20 nm/sec; accumulations, 10; response, 1 sec; bandwidth, 1 nm; path length, 0.1 cm). The α-helical content of each peptide is calculated by dividing the mean residue ellipticity by the reported value for a model helical decapeptide (Yang et al., Methods Enzymol. 130:208 (1986)).

Assays to Determine Melting Temperature (Tm): Cross-linked or the unmodified template peptides are dissolved in distilled H₂O or other buffer or solvent (e.g. at a final concentration of 50 μM) and Tm is determined by measuring the change in ellipticity over a temperature range (e.g. 4 to 95 ° C.) on a spectropolarimeter (e.g., Jasco J-710, Aviv) using standard parameters (e.g. wavelength 222 nm; step resolution, 0.5 nm; speed, 20 nm/sec; accumulations, 10; response, 1 sec; bandwidth, 1 nm; temperature increase rate: 1° C./min; path length, 0.1 cm).

In Vitro Protease Resistance Assays: The amide bond of the peptide backbone is susceptible to hydrolysis by proteases, thereby rendering peptidic compounds vulnerable to rapid degradation in vivo. Peptide helix formation, however, typically buries and/or twists and/or shields the amide backbone and therefore may prevent or substantially retard proteolytic cleavage. The peptidomimetic macrocycles of the present invention may be subjected to in vitro enzymatic proteolysis (e.g. trypsin, chymotrypsin, pepsin) to assess for any change in degradation rate compared to a corresponding uncrosslinked or alternatively stapled polypeptide. For example, the peptidomimetic macrocycle and a corresponding uncrosslinked polypeptide are incubated with trypsin agarose and the reactions quenched at various time points by centrifugation and subsequent HPLC injection to quantitate the residual substrate by ultraviolet absorption at 280 nm. Briefly, the peptidomimetic macrocycle and peptidomimetic precursor (5 mcg) are incubated with trypsin agarose (Pierce) (S/E ˜125) for 0, 10, 20, 90, and 180 minutes. Reactions are quenched by tabletop centrifugation at high speed; remaining substrate in the isolated supernatant is quantified by HPLC-based peak detection at 280 nm. The proteolytic reaction displays first order kinetics and the rate constant, k, is determined from a plot of In[S] versus time.

Peptidomimetic macrocycles and/or a corresponding uncrosslinked polypeptide can be each incubated with fresh mouse, rat and/or human serum (e.g. 1-2 mL) at 37° C. for, e.g., 0, 1, 2, 4, 8, and 24 hours. Samples of differing macrocycle concentration may be prepared by serial dilution with serum. To determine the level of intact compound, the following procedure may be used: The samples are extracted, for example, by transferring 100 μL of sera to 2 ml centrifuge tubes followed by the addition of 10 μL of 50% formic acid and 500 μL acetonitrile and centrifugation at 14,000 RPM for 10 min at 4+/−2° C. The supernatants are then transferred to fresh 2 ml tubes and evaporated on Turbovap under N₂<10 psi, 37° C. The samples are reconstituted in 100 μL of 50:50 acetonitrile:water and submitted to LC-MS/MS analysis. Equivalent or similar procedures for testing ex vivo stability are known and may be used to determine stability of macrocycles in serum.

In Vivo Protease Resistance Assays: A key benefit of peptide stapling is the translation of in vitro protease resistance into markedly improved pharmacokinetics in vivo.

In vitro Binding Assays: To assess the binding and affinity of peptidomimetic macrocycles and peptidomimetic precursors to acceptor proteins, a fluorescence polarization assay (FPA) can be used, for example. The FPA technique measures the molecular orientation and mobility using polarized light and fluorescent tracer. When excited with polarized light, fluorescent tracers (e.g., FITC) attached to molecules with high apparent molecular weights (e.g. FITC-labeled peptides bound to a large protein) emit higher levels of polarized fluorescence due to their slower rates of rotation as compared to fluorescent tracers attached to smaller molecules (e.g. FITC-labeled peptides that are free in solution).

Live Cell Detection: Cells are seeded and grown overnight. Serial dilutions of a stabilized peptide linked to a detectable label (e.g., a fluorescent label such lysine(TAMRA) or FITC) are then applied to the cells, and the cells are further incubated to allow binding to biomarker, e.g., a protein. Hoechst DNA dye can additionally be added to the cells to stain nuclei. Cells are then washed, and then confocal microscopy is used to determine the presence and localization (e.g., intracellular localization) of the labeled stabilized peptide over time.

Fixed Cell Detection: Cells are seeded and grown overnight. The cells are then washed in PBS (e.g., 2×), fixed using paraformaldehyde (e.g., 4% at room temperature), and then permeabilized (e.g., 0.5% Triton for 1 min). Hoechst DNA dye can additionally be added to the cells to stain nuclei. The cells can be rinsed, blocked in 3% BSA for 30 minutes, and serial dilutions of a stabilized peptide linked to a detectable label (e.g., a fluorescent label such as FITC, or a biotin label that can be recognized by adding streptavidin conjugated to a detection agent such as FITC, Cy5 [e.g., Cy5-streptavidin, ThermoFisher Scientific Cat# SA1011 for 30 minutes] or HRP) is transferred to the cells, and the cells are incubated to allow binding to biomarker, e.g., a protein. Confocal microscopy is used to detect the presence and localization of the stabilized peptide (direct fluorescence visualization) or light microscopy can be used based on colorimetric staining (HRP/peroxide exposure).

Methods of Detecting Proteins using Stabilized Peptides

### Methods of Detecting Proteins in Live Cells

Provided herein are methods of detecting a biomarker on, outside, or within a cell by contacting a cell or tissue with a stabilized peptide that binds to the biomarker, wherein the stabilized peptide is linked to a detectable label; and detecting the detectable label when the stabilized peptide is bound to the biomarker in the cell or tissue. In certain embodiments, the stabilized peptide binds to the biomarker on, outside, or within a live cell better and/or for a longer period of time relative to the peptide from which the stabilized peptide is derived or relative to a second stabilized peptide also derived from the same peptide.

In some embodiments, the stabilized peptide, e.g., a stapled or stitched peptide, can be linked to a detectable label. Once the stabilized peptide binds to a biomarker on, outside, or within a cell, the detectable label can be used to determine the location and/or relative amount of the biomarker. As used herein, a “detectable label” or “detectable moiety” refers to a label that is attached through covalent or non-covalent means to a stabilized peptide. In some embodiments, the detectable label provides a means for detection or quantitation of the stabilized peptide comprising the detectable label. In other embodiments, the detectable label provides a means for separating and/or purifying the stabilized peptide comprising the detectable moiety, e.g., the detectable label is an affinity tag (e.g., a FLAG-tag, His-Tag, Flu-tag, GFP-tag, biotin, etc.). In other embodiments, the detectable label provides a means for separating and/or purifying a biomarker, e.g., a protein, that is bound by a stabilized peptide comprising the detectable label. In some embodiments, the detectable label comprises a polypeptide (e.g., a GST-tag, a His-tag, a FLAG-tag, a myc-tag, or a HA-tag, a fluorescent protein (e.g., a GFP or a YFP), or a dye). In some embodiments, the detectable label comprises a radioactive label, a fluorescent label, a chemiluminescent label, a mass label, a charge label, an enzyme (e.g., an enzyme for which substrate converting activity of the enzyme is observed to reveal the presence of the stabilized peptide), a substrate for an enzyme, or a radioisotope. In some embodiments, the detectable label comprises fluorescein (FITC). In some embodiments, the detectable label comprises tetramethylrhodamine (TAMRA) azide, e.g., the stabilized peptide can comprise a lysine residue with the fluorescent dye TAMRA attached to it side chain amine. In some embodiments, the detectable label comprises a cyanine, e.g., Cy3 or Cy5. In some embodiments, the detectable label comprises biotin, such that the biotin can be detected using a labeled streptavidin (e.g., streptavidin-HRP). Detectable labels for use in the present invention may be attached to any part of the stabilized peptide, e.g., the N terminus, the C terminus, or anywhere in between (e.g., the middle of the peptide), so long as it does not disrupt binding to the biomarker or destabilize the peptide. In some embodiments, the detectable label is attached to the N-terminus of the stabilized peptide. In some embodiments, the detectable label is attached to the C-terminus of the stabilized peptide. In some embodiments, the stabilized peptide comprises one, two, three, four, five, six, seven, eight, nine, ten or more detectable labels. In some embodiments the detectable label is cleavable. In other embodiments, the detectable label is non-cleavable. In some embodiments, the detectable label is attached to the stabilized peptide via a linker. In some embodiments, the linker is cleavable. In other embodiments, the linker is non-cleavable.

The term “detectable label”, with regard to a stabilized peptide described herein, can encompass direct labeling of the stabilized peptide by coupling (i.e., physically linking) a detectable substance to the peptide or integrating a detectable substance into the peptide, as well as indirect labeling of the stabilized peptide by reactivity with another reagent (a secondary reagent) that is directly or indirectly labeled with a detectable substance. An example of direct labeling includes covalently attaching a fluorescent label (e.g., a lysine(TAMRA)) to the stabilized peptide. An example of indirect labeling includes detection of a labeled stabilized peptide using a secondary reagent, e.g., a fluorescently-labeled secondary antibody that binds to the peptide (e.g., binds to a portion of the peptide or to the label attached to the peptide). In another example, a stabilized peptide comprising biotin is detected using a secondary reagent comprising a fluorescently-labeled streptavidin that binds to the biotin attached to the peptide. In another example, a stabilized peptide is detected using a horseradish peroxidase (HRP)-conjufated secondary antibody that binds to the peptide (e.g., binds to a portion of the peptide or to the label attached to the peptide), and the HRP-conjugated secondary antibody is detected using a chemical (e.g., comprising an HRP substrate) that detects HRP. In another example of indirect labeling, a stabilized peptide comprising biotin is detected using a secondary reagent comprising a HRP-streptavidin that binds to the biotin attached to the peptide, and the HRP-streptavidin is detected using a chemical (e.g., comprising an HRP substrate) that detects HRP.

Thus, it will be understood to the skilled artisan that methods involving indirect labeling of the stabilized peptide involve additional steps comprising: (a) contacting the cells or tissue with a directly labeled secondary reagent (e.g., a fluorescently-labeled secondary antibody that binds to the stabilized peptide (e.g., binds to a portion of the peptide or to the label attached to the peptide) or a fluorescently-labeled streptavidin that binds to biotin attached to the peptide), (b) after a period of time to allow the directly labeled secondary reagent to interact with (e.g., bind to) the stabilized peptide on, outside, or within the cell, the cell or tissue is washed to reduce nonspecific binding of the secondary reagent, and (c) detecting the directly labeled secondary reagent. Alternatively, methods involving indirect labeling of the stabilized peptide involve additional steps comprising: (a) contacting the cells or tissue with an indirectly labeled secondary reagent (e.g., a HRP-conjugated secondary antibody that binds to the stabilized peptide (e.g., binds to a portion of the peptide or to the label attached to the peptide) or a HRP-streptavidin that binds to biotin attached to the peptide), (b) after a period of time to allow the directly labeled secondary reagent to interact with (e.g., bind to) the stabilized peptide on, outside, or within the cell, the cell or tissue is washed to reduce nonspecific binding of the secondary reagent, (c) contacting the cells or tissue with a chemical (e.g., comprising an HRP substrate) that detects the secondary reagent (e.g., the HRP), (d) after a period of time to allow the chemical to interact with (e.g., bind to) the secondary reagent on, outside, or within the cell, the cell or tissue is washed to reduce nonspecific binding of the chemical; and (e) detecting the indirectly labeled secondary reagent.

In one embodiment, the antibody is labeled, e.g. a radio-labeled, chromophore-labeled, fluorophore-labeled, or enzyme-labeled antibody. In another embodiment, an antibody derivative (e.g. an antibody conjugated with a substrate or with the protein or ligand of a protein-ligand pair {e.g. biotin-streptavidin}), or an antibody fragment (e.g. a single-chain antibody, an isolated antibody hypervariable domain, etc.) which binds specifically with a marker of the invention.

In some embodiments, a stabilized peptide, e.g., a stapled or stitched peptide, can be used to detect a biomarker on, outside, or within living cells. For example, a living cell or tissue can be contacted with a stabilized peptide comprising a detectable label that binds to a particular biomarker. The stabilized peptide is given a period of time to bind to the biomarker in a living cell, e.g., 2 minutes, 5 minutes, 10 minutes, 15 minutes, 20 minutes, 25 minutes, 30 minutes, 35 minutes, 40 minutes, 45 minutes, 50 minutes, 55 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, 12 hours, or 24 hours or more. Once the stabilized peptide is bound to the biomarker, the presence or absence of the biomarker, the quantity or amount of the biomarker, and/or the localization (e.g., intracellular localization) of the biomarker is determined by detecting the detectable label linked to the stabilized peptide on, outside, or within the living cell. In some embodiments, a living cell is contacted with a stabilized peptide, as described herein, and then the presence, amount, and/or localization of the biomarker is monitored at set intervals of time, e.g., every 5 seconds, 10 seconds, 15 seconds, 30 seconds, 45 seconds, 1 minute, 5 minutes, 10 minutes, 15 minutes, 20 minutes, 25 minutes, 30 minutes, 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 24 hours, 30 hours, 36 hours, 42 hours, or 48 hours or more. In some embodiments, the presence, amount, and/or localization of the biomarker can be determined over a time course of 10 minutes, 15 minutes, 20 minutes, 25 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 24 hours, 36 hours, or 48 hours or more.

Numerous methods and assays are known in the art for detecting a biomarker-binding agent comprising a detectable label in a living cell. By way of example, a stabilized peptide comprising a fluorescent label, e.g., a stapled and/or stitched peptide comprising lysine(TAMRA), can be detected in one or more living cells using fluorescence microscopy, e.g., fluorescence confocal microscopy.

In some embodiments, a stabilized peptide linked to a detectable label can be used to screen for the presence and/or amounts of a biomarker in numerous cells using high throughput assays. For example, a stabilized peptide linked to a fluorescent label can be used to detect a biomarker and/or determine the amounts of the biomarker in a population of living cells using fluorescence-activated cell sorting (FACS).

In some embodiments, a stabilized peptide linked to a detectable label can be used to detect a biomarker on, outside, or within a living cell in culture, e.g., a cell of a tissue culture cell line. In some embodiments, a stabilized peptide linked to a detectable label can be used to detect a biomarker on, outside, or within a living cell that has been isolated from a subject, e.g., a patient with a disease or suspected of having a disease. For example, a stabilized peptide linked to a detectable label can be used to detect a biomarker on, outside, or within a primary cell that has been isolated from a subject and cultured. In some embodiments, a stabilized peptide linked to a detectable label can be used to detect a biomarker on, outside, or within a cell of a sample that has been isolated from a subject. The term “sample” includes any body fluid (e.g., blood fluids (e.g., blood, serum or plasma), lymph, cerebrospinal fluid, gynecological fluids (e.g., vaginal fluid), semen, cystic fluid, urine, ocular fluids, pleural fluid, fluids collected by bronchial lavage and/or peritoneal rinsing, and a secretion (e.g., sweat or saliva)), cell, or tissue from a subject. In one embodiment, the tissue or cell is removed from the subject. In another embodiment, the tissue or cell is present within the subject. Other samples include serum, cerebrospinal fluid, feces, sputum, secretions, and cell extracts. In one embodiment the sample is a blood sample. In one embodiment, the sample contains protein molecules from the subject. In one embodiment, the sample is a tumor or tissue sample, e.g., a tumor or tissue biopsy sample.

The stabilized peptides described herein can be used to detect a biomarker on, outside, or within a bacterial cell, a fungal cell, a protest, an animal cell, or a plant cell. In some embodiments, the cell is an avian cell, e.g., a chicken cell. In some embodiments, the cell is a reptilian cell. In some embodiments, the cell is a mammalian cell, e.g., a cell of a mouse, rat, dog, cat, rabbit, alpaca, camelid, horse, cow, sheep, goat, or primate. In some embodiments, the cell is a human cell.

### Methods of Detecting Proteins in Fixed Cells

Fixation solutions introduce cross links between amino acids, which has the potential of destroying the binding of an antibody to its antigen. In contrast, stabilized peptide reagents are particularly useful in fixed cells, as they can be designed to bind the target protein at sites that are undisturbed by the fixation process. In particular, because the binding site on the stabilized peptide's target protein is mostly hydrophobic, the binding site is less affected by fixation solutions. As a result, the binding site for the stabilized peptide reagent is sufficiently preserved to enable the “helix-in-groove” interaction between the binding site and a stabilized helical peptide reagent. Thus, in some embodiments, a stabilized peptide, e.g., a stapled or stitched peptide, can be used to detect a biomarker on, outside, or within a cell that has been fixed for immunological staining (e.g., a fixed tissue sample). In certain embodiments, the stabilized peptide binds to the biomarker on the fixed cell better and/or for a longer period of time relative to the peptide from which the stabilized peptide is derived. In certain embodiments, the stabilized peptide binds to the biomarker on the fixed cell better and/or for a longer period of time or relative to a second stabilized peptide also derived from the same peptide.

In some embodiments, the stabilized peptide, e.g., a stapled or stitched peptide, can be linked to a detectable label. Once the stabilized peptide binds to a biomarker on, outside, or within a fixed cell (e.g., a fixed tissue sample), the detectable label can be used to determine the location and/or relative amount of the biomarker. As described above, a “detectable label” or “detectable moiety” refers to a label that is attached through covalent or non-covalent means to a stabilized peptide. In some embodiments, the detectable label provides a means for detection or quantitation of the stabilized peptide comprising the detectable label. In other embodiments, the detectable label provides a means for separating and/or purifying the stabilized peptide comprising the detectable moiety, e.g., the detectable label is an affinity tag (e.g., a FLAG-tag, His-Tag, Flu-tag, GFP-tag, biotin, etc.). In other embodiments, the detectable label provides a means for separating and/or purifying a biomarker, e.g., a protein, that is bound by a stabilized peptide comprising the detectable label. In some embodiments, the detectable label comprises a polypeptide (e.g., a GST-tag, a His-tag, a FLAG-tag, a myc-tag, or a HA-tag, a fluorescent protein (e.g., a GFP or a YFP), or a dye). In some embodiments, the detectable label comprises a radioactive label, a fluorescent label, a chemiluminescent label, a mass label, a charge label, an enzyme (e.g., an enzyme for which substrate converting activity of the enzyme is observed to reveal the presence of the stabilized peptide), a substrate for an enzyme, or a radioisotope. In some embodiments, the detectable label comprises fluorescein (FITC). In some embodiments, the detectable label comprises tetramethylrhodamine (TAMRA) azide, e.g., the stabilized peptide can comprise a lysine residue with the fluorescent dye TAMRA attached to it side chain amine. In some embodiments, the detectable label comprises a cyanine, e.g., Cy3 or Cy5. In some embodiments, the detectable label comprises biotin, such that the biotin can be detected using a labeled streptavidin (e.g., streptavidin-HRP). Detectable labels for use in the present invention may be attached to any part of the stabilized peptide, e.g., the N terminus, the C terminus, or anywhere in between (e.g., the middle of the peptide), so long as it does not disrupt binding to the biomarker or destabilize the peptide. In some embodiments, the detectable label is attached to the N-terminus of the stabilized peptide. In some embodiments, the detectable label is attached to the C-terminus of the stabilized peptide. In some embodiments, the stabilized peptide comprises one, two, three, four, five, six, seven, eight, nine, ten or more detectable labels. In some embodiments the detectable label is cleavable. In other embodiments, the detectable label is non-cleavable. In some embodiments, the detectable label is attached to the stabilized peptide via a linker. In some embodiments, the linker is cleavable. In other embodiments, the linker is non-cleavable.

As described above, the term “detectable label”, with regard to a stabilized peptide described herein, can encompass direct labeling of the stabilized peptide by coupling (i.e., physically linking) a detectable substance to the peptide or integrating a detectable substance into the peptide, as well as indirect labeling of the stabilized peptide by reactivity with another reagent (a secondary reagent) that is directly or indirectly labeled with a detectable substance. An example of direct labeling includes covalently attaching a fluorescent label (e.g., a lysine(TAMRA)) to the stabilized peptide. An example of indirect labeling includes detection of a labeled stabilized peptide using a secondary reagent, e.g., a fluorescently-labeled secondary antibody that binds to the peptide (e.g., binds to a portion of the peptide or to the label attached to the peptide). In another example, a stabilized peptide comprising biotin is detected using a secondary reagent comprising a fluorescently-labeled streptavidin that binds to the biotin attached to the peptide. In another example, a stabilized peptide is detected using a horseradish peroxidase (HRP)-conjufated secondary antibody that binds to the peptide (e.g., binds to a portion of the peptide or to the label attached to the peptide), and the HRP-conjugated secondary antibody is detected using a chemical (e.g., comprising an HRP substrate) that detects HRP. In another example of indirect labeling, a stabilized peptide comprising biotin is detected using a secondary reagent comprising a HRP-streptavidin that binds to the biotin attached to the peptide, and the HRP-streptavidin is detected using a chemical (e.g., comprising an HRP substrate) that detects HRP.

A cell or a tissue sample can be fixed using common techniques known in the art for immunological staining (see, e.g., the Examples section below). After the cell or tissue is fixed, it is contacted with a stabilized peptide linked to a detectable label. After a period of time to allow the stabilized peptide to bind to a biomarker on, outside, or within a cell, the cell or tissue is washed to reduce nonspecific binding of the stabilized peptide. Optionally, if the stabilized peptide is indirectly labeled, the method further comprises one or more additional steps to detect the indirectly labeled stabilized peptide, e.g., as described below. The detectable label linked to the peptide is detected (directly or indirectly) to determine the presence or absence of the biomarker, the amount or quantity of the biomarker, and/or the localization (e.g., intracellular localization) of the biomarker. In some embodiments, the stabilized peptide is given a period of time to bind to the biomarker in a fixed cell or tissue, e.g., 2 minutes, 5 minutes, 10 minutes, 15 minutes, 20 minutes, 25 minutes, 30 minutes, 35 minutes, 40 minutes, 45 minutes, 50 minutes, 55 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, 12 hours, or 24 hours or more.

It will be understood to the skilled artisan that methods involving indirect labeling of the stabilized peptide involve additional steps comprising: (a) after the period of time to allow the stabilized peptide to bind to the biomarker on, outside, or within the cell, contacting the cell or tissue with a directly labeled secondary reagent (e.g., a fluorescently-labeled secondary antibody that binds to the stabilized peptide (e.g., binds to a portion of the peptide or to the label attached to the peptide) or a fluorescently-labeled streptavidin that binds to biotin attached to the peptide), (b) after a period of time to allow the directly labeled secondary reagent to interact with (e.g., bind to) the stabilized peptide on, outside, or within the cell, the cell or tissue is washed to reduce nonspecific binding of the secondary reagent, and (c) detecting the directly labeled secondary reagent. Alternatively, methods involving indirect labeling of the stabilized peptide involve additional steps comprising: (a) after the period of time to allow the stabilized peptide to bind to the biomarker on, outside, or within the cell, contacting the cell or tissue with an indirectly labeled secondary reagent (e.g., a HRP-conjugated secondary antibody that binds to the stabilized peptide (e.g., binds to a portion of the peptide or to the label attached to the peptide) or a HRP-streptavidin that binds to biotin attached to the peptide), (b) after a period of time to allow the directly labeled secondary reagent to interact with (e.g., bind to) the stabilized peptide on, outside, or within the cell, the cell or tissue is washed to reduce nonspecific binding of the secondary reagent, (c) contacting the cell or tissue with a chemical (e.g., comprising an HRP substrate) that detects the secondary reagent (e.g., the HRP), (d) after a period of time to allow the chemical to interact with (e.g., bind to) the secondary reagent on, outside, or within the cell, the cell or tissue is washed to reduce nonspecific binding of the chemical; and (e) detecting the indirectly labeled secondary reagent. In some embodiments, the period of time to allow the secondary reagent to interact with (e.g., bind to) the stabilized peptide in the fixed cell or tissue is 2 minutes, 5 minutes, 10 minutes, 15 minutes, 20 minutes, 25 minutes, 30 minutes, 35 minutes, 40 minutes, 45 minutes, 50 minutes, 55 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, 12 hours, or 24 hours or more. In some embodiments, the period of time to allow the chemical to interact with (e.g., bind to) the secondary reagent in the fixed cell or tissue is 2 minutes, 5 minutes, 10 minutes, 15 minutes, 20 minutes, 25 minutes, 30 minutes, 35 minutes, 40 minutes, 45 minutes, 50 minutes, 55 minutes, 1 hour, 1.5 hours, 2 hours, 2.5 hours, 3 hours, 3.5 hours, 4 hours, 4.5 hours, 5 hours, 6 hours, 7 hours, 8 hours, 9 hours, 10 hours, 11 hours, 12 hours, or 24 hours or more.

Numerous methods and assays are known in the art for detecting a biomarker-binding agent comprising a detectable label in a fixed cell or tissue. By way of example, a stabilized peptide comprising a fluorescent label, e.g., a stapled and/or stitched peptide comprising lysine(TAMRA), can be detected in one or more fixed cells using fluorescence microscopy, e.g., fluorescence confocal microscopy, or FACs. As another example, a stabilized peptide comprising a biotin label, e.g., a stapled and/or stitched peptide comprising biotin, can be detected in one or more fixed cells by contacting the fixed cells with a directly labeled streptavidin (e.g., a fluorescently labeled streptavidin).

In some embodiments, a stabilized peptide linked to a detectable label can be used to screen for the presence and/or amounts of a biomarker in numerous cells using high throughput assays. For example, a stabilized peptide linked to a fluorescent label can be used to detect a biomarker and/or determine the amounts of the biomarker in a population of fixed cells using fluorescence-activated cell sorting (FACS).

A skilled artisan would understand that most cells can be fixed and immunologically stained to detect a particular biomarker if a detectable biomarker-binding agent is available that can specifically bind to the biomarker. The stabilized peptides described herein can be used to detect a biomarker on, outside, or within a fixed bacterial cell, fungal cell, protest, animal cell, or plant cell. In some embodiments, the cell is an avian cell, e.g., a chicken cell. In some embodiments, the cell is a reptilian cell. In some embodiments, the cell is a mammalian cell, e.g., a cell of a mouse, rat, dog, cat, rabbit, alpaca, camelid, horse, cow, sheep, goat, or primate. In some embodiments, the cell is a human cell. In some embodiments, the fixed cells or tissue are from a biopsy. In some embodiments, the biopsy is a cancer biopsy (e.g., of a tumor).

### Methods of Detecting Proteins in Solution

In certain instances, the disclosure features methods of using a stabilized peptide (e.g., stapled and/or stitched peptide) to detect a biomarker in a solution. The stabilized peptide binds to the biomarker in solution better and/or for a longer period of time relative to the peptide from which the stabilized peptide is derived. In certain embodiments, the stabilized peptide binds to the biomarker in solution better and/or for a longer period of time relative to a second stabilized peptide also derived from the same peptide. The stabilized peptide can be linked to a detectable label. The liquid solution can be e.g., blood, serum, plasma, urine, mucous, cerebrospinal fluid, a lavage, pleural fluid, vaginal fluid, semen, peritoneal fluid, or a secretion (e.g., sweat or saliva). In certain instances, the liquid solution is blood. In certain instances, the stabilized peptide can be linked to a solid carrier. In some instances, the solid carrier is a magnetic bead. In some instances, the solid carrier is a biodegradable bead. In some instances, the solid carrier is a polymer bead. In some instances in which the stabilized peptide is linked to a solid carrier, the stabilized peptide is purified from the solution and the amount of the stabilized peptide is assayed or quantified.

Non-limiting examples of detectable labels that can be used in the methods of detecting proteins in solution described herein are provided herein (see, e.g., the Methods of Detecting Proteins in Live Cells section above and the Examples below).

Methods of using Stabilized Peptides to Detect Disease

Diagnostic antibodies are not available for many proteins with known links to disease or that are suspected of being associated with disease. The lack of detectable antibodies for some proteins has impeded the acquisition of information about the function of these proteins and the development of therapeutics. Thus, the stabilized peptides described herein fill a critical unmet need for reagents that can precisely bind to, detect, and monitor the amounts of and localization of proteins linked to disease for which there are no suitable or effective diagnostic reagents. Moreover, the stabilized peptides described herein have several advantages compared to diagnostic antibody technology. For example, stabilized peptides can be designed and optimized from known protein-protein binding domains; they can be synthesized quickly, at high purity, and relatively cheaply compared to antibodies; they have a long shelf life; they can display a high affinity and specificity for protein targets; and they can be used to detect and monitor target proteins in live cells.

In certain aspects, the present invention provides methods and compositions for detecting or diagnosing a disease or a disorder using one or more stabilized peptides described herein. In some embodiments, a cell or a sample (e.g., a tissue sample) is isolated from a subject, e.g., a human subject suspected of having a disease, and then contacted with a stabilized peptide described herein that is designed to bind to a particular biomarker in the cell or sample whose presence, absence, particular level or amount, and/or localization can be used to help determine whether the subject has the disease. After the stabilized peptide is allowed to bind to the biomarker in the cell or sample, the stabilized peptide and any biomarker bound by the peptide is detected (directly or indirectly). The presence of biomarker, level or amount of biomarker, and/or localization (e.g., intracellular or extracellular localization) of biomarker can then be analyzed and used to determine whether the subject has the disease. Non-limiting examples of detectable labels that can be used in the methods of detecting diseases described herein are provided herein (see, e.g., the Methods of Detecting Proteins in Live Cells section above and the Examples below).

In some embodiments, the stabilized peptides (e.g., stapled and/or stitched peptide linked to a detectable label) and methods described herein can be used to determine the level or amount of the biomarker in one or more cells or samples collected from a subject. In some embodiments, a biomarker level above or below a certain threshold level can indicate whether the subject has a disease, such that the biomarker can be used as a biomarker for disease. The methods include determining the level of one or more biomarkers in at least one cell or sample collected from the subject, and comparing this level to the level of the same one or more biomarkers in at least one control cell or sample. A difference in the level (e.g., higher or lower) of the one or more biomarkers in the at least one cell or sample from the subject as compared to the level of the one or more biomarkers in the at least one control cell or sample indicates that the subject has a disease.

Essentially any technical means established in the art for detecting the level a marker of the invention at either the nucleic acid or protein level, can be used to determine the level a marker of the invention as discussed herein.

In any of the methods described herein, the level of a biomarker in a cell or sample obtained from a subject can be determined using well-known techniques and methods which allow for the detection and measurement of a labeled biomarker-binding agent that is bound to the biomarker in a live or fixed cell. Non-limiting examples of such methods that can be used to detect and measure the level of a biomarker bound by a stabilized peptide described herein include immunological methods for detection of the labeled stabilized peptide, such as immunofluorescence microscopy, immunohistochemistry, flow cytometry, and the like, and combinations thereof. In some embodiments, immunofluorescence signals in the cell obtained from a subject are measured and quantified, and compared to immunofluorescence signals in a control cell or sample. In some embodiments, binding of the stabilized peptide to the biomarker, e.g., a protein, is used to purify or extract the biomarker from the cell or sample (e.g., by immunoprecipitation), and then biomarker is then quantified using a technique well known in the art, e.g., immunoblotting, Western blotting, mass spectrometry, capillary electrophoresis, enzyme linked immunosorbent assays (ELISAs, and the like, and combinations thereof).

A biomarker is a biomolecule which can be differentially present in a sample taken from a subject of one phenotypic status (e.g., having a disease) as compared with another phenotypic status (e.g., not having the disease). A biomarker can be differentially present between different phenotypic statuses if the mean or median level, e.g., expression level, of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. As such, they are useful for detecting or diagnosing disease, or for determining the therapeutic efficacy of a treatment for disease, e.g., the efficacy of a drug therapeutic for reducing disease. In some embodiments, the stapled peptides and methods described herein are used to detect a biomarker or a protein in a cell, e.g., a live or fixed cell, that is a biomarker for a particular disease.

The “level of a biomarker” refers to an amount of the biomarker, e.g., a biomarker, present in a sample being tested. A level of a biomarker may be either in absolute level or amount (e.g., μg/ml) or a relative level or amount (e.g., relative intensity of signals).

A “lower level” or a “decrease in the level” of a biomarker refers to a level of the biomarker in a test sample that is less than the standard error of the assay employed to assess the level of the marker, and preferably at least twice, and more preferably three, four, five, six, seven, eight, nine, or ten or more times less than the level of the biomarker in a control sample (e.g., a sample from a subject with a wild-type phenotype or a subject without particular disease and/or, the average level of the biomarker in several control samples). In some embodiments, the stapled peptides and methods described herein are used to detect the level of a biomarker in a cell collected from a subject, e.g., a live or fixed cell. The biomarker is a biomarker for a particular disease, and the level of the biomarker in the cell is used to determine the likelihood that subject has a disease.

The term “control level” refers to an accepted or pre-determined level of a biomarker, e.g., a protein, which is used to compare the level of the biomarker, e.g., the protein, in a sample collected from a subject. In one embodiment, the control level of a biomarker is based the level of the biomarker in a cell or sample from a subject having no disease. In another embodiment, the control level of a biomarker is based the level of the biomarker in a cell or sample from a subject known to have disease. In another embodiment, the control level of a biomarker in a cell or sample is based on the level of the biomarker previously determined in a cell or sample from the subject. In yet another embodiment, the control level of a biomarker is based on the level of the biomarker in a cell or sample from the subject prior to the administration of a therapy for a disease. Alternatively, population-average values for the “control” level of a biomarker may be used, e.g., average biomarker level derived from biomarker levels in many samples collected from many subjects. In other embodiments, the “control” level of a biomarker may be determined by determining the level of the biomarker in a cell or sample obtained from the subject before the onset of disease, from an archived cell or sample.

In certain embodiments, a subject is suspected of having a disease that is associated with one or more of the biomarkers listed in Table 1. In some embodiments, the methods include determining the level of one or more biomarkers listed in Table 1 in a cell or sample obtained from a subject, and comparing the level of the one or more biomarkers in the cell or sample from the subject with a level of the one or more biomarkers in a control cell or sample (e.g., a cell or sample from a healthy subject), wherein a difference in the level of the one or more biomarkers in the cell or sample from the subject as compared to the level of the one or more biomarkers in the control cell or sample indicates that the subject has a disease.

In some embodiments, the stabilized peptides and methods described herein can be used to diagnose a disease in a subject suspected of having a cancer, an autoimmune disease, an inflammatory disease, an infectious diseases, and/or a neurologic disease. By way of example, the stabilized peptides and methods described herein can be used detect or diagnose a melanoma, a leukemia, lymphoma, or other hematologic malignancy or solid tumor. In certain instances, the solid tumor is a melanoma, a breast cancer or a lung cancer. In some embodiments, the stabilized peptide and methods described herein can be used to detect or diagnose an autoimmune disease or an inflammatory disease. In certain instances, the autoimmune disease is autoimmune colitis, thyroiditis, arthritis, nephritis, dermatitis, vasculitis, system lupus erythematosus, diabetes, or Sjogren's disease. In some instances, the inflammatory disease is asthma, psoriasis, inflammatory colitis, thyroiditis, arthritis, nephritis, dermatitis, or vasculitis. In some embodiments, the stabilized peptides and methods described herein can be used to detect or diagnose an infectious disease or a disorder caused by a pathogen in a subject, e.g., influenza, HIV, tuberculosis, or streptococcus. In some embodiments, the stabilized peptides and methods described herein can be used to detect or diagnose a neurologic disease, such as Alzheimer's, Huntington's, Parkinson's, or other neurologic diseases.

In some embodiments, a stabilized peptide described herein comprising a detectable label is used to detect the presence of a biomarker, e.g., a protein, and/or quantify the amount of biomarker, e.g., a protein, in a tumor biopsy sample isolated from a subject, e.g., a subject with cancer or suspected of having cancer. The presence of the biomarker and/or the relative amount of biomarker in the tumor biopsy sample compared to the presence and/or amount of the biomarker in a control sample, e.g., a tissue sample collected from a healthy subject, can be used to determine whether the subject has cancer or is at an elevated risk of developing cancer.

The stabilized peptides and methods described herein can be used in conjunction with any other method and/or composition used by the skilled practitioner to diagnose, prognose, and/or monitor a disease or disorder. For example, a stabilized peptide described herein can be used to detect a biomarker, e.g., a protein, associated with disease in a cell in conjunction with another reagent that can be used to detect and/or measure the same or another molecular marker of disease, e.g., another biomarker or protein associated with the disease. The stabilized peptides and methods described herein can also be performed in conjunction with any clinical measurement of the disease or disorder known in the art including clinical evaluation, serological evaluation, pathological evaluation, and/or detection (and quantification, if appropriate) of other molecular markers of the disease.

The stabilized peptides and methods described herein can also be used to monitor the progression of a disease in a subject. In some embodiments, cells or samples, e.g., tissues, can be collected from a subject over time, e.g., a cell or sample can be collected from a subject once a day, once a week, once every 2 weeks, once a month, once every 2 months, once every 3 months, once every 4 months, once every 5 months, or once every 6 months, or more. At each time point, a stabilized peptide can be used to determine the presence, levels or amounts, and/or localization of the biomarker in the cell or sample collected from the subject. Qualitative and/or quantitative modulation of the presence, levels, and/or localization of the biomarker in the cells or samples collected over time can be used to determine the progression of disease in the subject.

Methods of using Stabilized Peptides to Monitor the Efficacy of a Disease Treatment

The stabilized peptides and methods disclosed herein can be used to monitor the efficacy of a therapy or treatment for a disease is a subject. In some embodiments, a detectable stabilized peptide can be used to determine the presence, levels or amounts, and/or localization of a biomarker or protein in cells or samples collected from a subject over time, including in cells and samples collected before treatment of the subject with a therapeutic, during treatment with the therapeutic, and/or after the cessation of treatment with the therapeutic. A qualitative and/or quantitative modulation in the presence, levels or amounts, and/or localization of the biomarker, e.g., a protein, in the cells or samples collected from the subject during the course of treatment for the disease can be used to determine whether the treatment is effective, e.g., changes in biomarker or protein presence, levels and/or localization towards a more wild-type phenotype (e.g., as in a healthy subject) during the course of treatment indicates that the therapy is effective.

### Kits for Detecting Proteins

A kit is any manufacture (e.g. a package or container) comprising at least one stabilized peptide reagent for specifically detecting a biomarker, e.g., an antigen or protein, as described herein. The manufacture is promoted, distributed, or sold as a unit for performing the methods described herein. The kits include means for determining the presence at least one biomarker, e.g., a least one antigen or protein, and instructions for using the kit. In certain embodiments, the kit may include a secondary detection agent, e.g., a secondary antibody with a detectable label, such as a fluorescent label, that is used to detect the stabilized peptide in a cell or sample. In some embodiments, the kit comprises instructions for detecting and determining the presence of a biomarker, e.g., an antigen or protein, measuring and/or quantitating the levels of the biomarker, e.g., the antigen or protein, and/or detecting and/or monitoring the localization of the biomarker, e.g., the antigen or protein, on, outside, or within a live cell or sample or a fixed cell or sample.

The kits can optionally comprise additional components useful for performing the methods described herein. By way of example, the kits may comprise reagents for obtaining a sample, e.g., a cell or tissue, from a subject, a control sample, one or more sample compartments, an instructional material which describes performance of a method described herein, and specific controls/standards. The kits described herein may also comprise reagents for culturing a sample, e.g., a cell or tissue, obtained from a subject.

The reagents for determining the level of one or more biomarkers can include, for example, buffers or other reagents for use in an assay for evaluating the level of one or more biomarkers on, outside, or within a live and/or fixed cell or tissue. The instructions can be, for example, printed instructions for performing the assay for evaluating the level of one or more biomarkers.

The reagents for isolating a sample from a subject can comprise one or more reagents that can be used to obtain a cell, tissue or fluid from a subject, such as means for obtaining saliva, blood or a biopsy sample.

In some embodiments, the methods and kits as described herein can be used as a companion diagnostic for a particular type of therapy or a particular therapeutic agent. For example, the methods and kits described herein can be used to determine if a particular therapy is suitable for treating a particular subject, e.g., a subject suspected of having a particular disease. In some embodiments, the methods and kits described herein can be used to determine if a therapy being administered to a subject having a known disease is effective in treating that disease. Thus, the methods and kits described herein can be used to assess and shape clinical decision making.

In one illustrative example, the methods and kits described herein can be used to determine whether a subject has a p53-associated cancer that overexpresses MDM2 and/or MDMX, and would thus be eligible for treatment with a therapeutic stabilized peptide that binds to MDM2 and/or MDMX, e.g., the ATSP-7041 stapled peptide as described in Chang et al., Proc. Nat'l Acad. Sci. (USA), 2013 110(36):E3445-54, herein incorporated by reference in its entirety, and/or the derived clinical agent, ALRN-6924. The ASTP-7041 and ALRN-6924 peptides bind to and inhibit MDM2 and MDMX, which activates the p53 pathway in tumors. One or more stabilized peptides as described herein can be designed to bind to MDM2 and/or MDMX in a cancerous cell or tumor sample collected from a subject with cancer or suspected of having a cancer. If MDM2 and/or MDMX levels are elevated in the cancerous cell or tumor sample collected from the subject relative to the levels in a control cell or sample (e.g., a cell or sample collected from a subject without cancer), then the subject is a candidate for treatment with the ASTP-7041 or ALRN-6924 stapled peptide.

In some embodiments, the methods and kits described herein can be used to determine whether a subject has a cancer that overexpress MCL-1, and would thus be eligible for treatment with a therapeutic that binds to MCL-1 and kills MCL-1-dependent cancer cells, e.g., the 563845 small molecule as described in Kotschy et al., Nature, 2016, 538(7626):477-482, herein incorporated by reference in its entirety. A stabilized peptide as described herein can be designed to bind to MCL-1 in a cancerous cell or tumor sample collected from a subject known to have a cancer or suspected of having a cancer. In some instances, the stabilized peptide can be a peptide as described in Stewart et al., Nat. Chem. Biol., 2010, 6(8):595-601 or Rezaei-Araghi et al., Proc. Natl. Acad. Sci. U.S.A., 2018, 115(5):E886-E895, which are herein incorporated by reference in their entirety. If MCL-1 levels are elevated in the cancerous cell or tumor sample collected from the subject relative to the levels in a control cell or sample (e.g., a cell or sample collected from a subject without cancer), then the subject is a candidate for treatment with the S63845.

In some embodiments, the methods and kits described herein can be used to determine whether a subject has a cancer that overexpress BFL-1, and would thus be eligible for treatment with a therapeutic that binds to BFL-1 and kills BFL-1-dependent cancer cells. A stabilized peptide as described herein can be designed to bind to BFL-1 in a cancerous cell or tumor sample collected from a subject known to have a cancer or suspected of having a cancer. In some instances, the stabilized peptide can be a peptide as described in Huhn et al., Cell Chem. Biol., 2016, 9:1123-1134 or Harvey et al., Structure, 2018, 26(1):153-160, which are herein incorporated by reference in their entirety. If BFL-1 levels are elevated in the cancerous cell or tumor sample collected from the subject relative to the levels in a control cell or sample (e.g., a cell or sample collected from a subject without cancer), then the subject is a candidate for treatment with a BFL-1 inhibitor.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1

### Production of Stabilized Peptides for Biomarker Detection

Peptide reagents are attractive candidates for use in detecting biomarkers on, outside, or within cells. However, removal of peptide sequences from their native protein environments can cause them to lose their secondary structure, often rapidly, thereby eliminating their ability to bind to target biomarkers in cells. To overcome this obstacle, peptides can be designed to incorporate particular chemical constraints, such as staples and/or stitches (e.g., hydrocarbon staples), that serve to reinforce their secondary structure, thereby stabilizing their ability to bind biomarkers. The process of tailoring such “stabilized peptides” for particular biomarker targets can be used to create diagnostic reagents with fluorescent and/or affinity tags for stably detecting the particular biomarkers in fixed or live cells, and with notably more efficient methodologies.

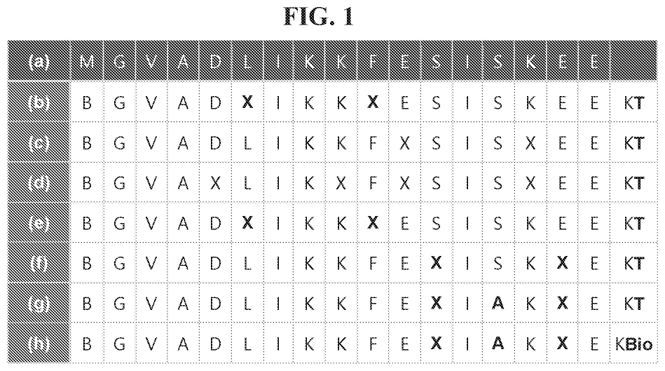

As a proof of concept, hydrocarbon staples were introduced at different locations in a peptide with known binding attributes for a target protein in order to identify stapled peptides with enhanced stability, cell penetrance, and that retain or enhance target binding. As shown in FIG. 1 (row (a)), a peptide composed of 17 amino acids from the N-terminus of the S. cerevisiae Abp140 protein that binds to actin filaments was chosen for modification. In the native protein, this peptide has an alpha-helical structure that is a recognition motif for actin. At least one all-hydrocarbon staple was introduced at various locations of the peptide to stabilize the alpha-helical recognition motif to produce stapled peptides markers for actin (see rows (b)-(g) in FIG. 1; the location of hydrocarbon staples in the peptide sequence is demarcated by an X, wherein X is (S)-2-(4′-pentenyl) alanine). These stapled peptides were linked to a fluorescent tag (5(6)-TAMRA (5-(and-6)-Carboxytetramethylrhodamine)), and then tested for actin-binding in mouse myoblast cells (C2C12 cell line, ATCC® CRL-1772™). Cells at a density of 10,000 cells/well were seeded in 96 well plates using DMEM media and incubated overnight at 37° C. For purposes of live cell imaging, serial dilutions of each peptide in the same culturing medium were then transferred to the cells, and the cells were incubated at 37° C. After 3 hours of incubation, cells were washed with PBS to remove extra peptide reagent and fluorescence microscopy was then used to assess actin binding by the peptides, and to determine overall peptide stability. In the case of fixed cell imaging, cells were initially permeabilized and fixed before the addition of the stapled peptide reagent. The stapled peptide that retained actin binding over the longest period of time, as determined by fluorescence in cells, was selected as having hydrocarbon staple(s) at the optimal sequence locations for improving peptide stability for biomarker detection.

Peptide (f), as shown in FIG. 1 (row (f)), exhibited actin binding for the longest period of time, and was therefore selected as being the most stable stapled peptide marker for actin. A serine to alanine substitution (514A) was then introduced in peptide (f) to produce peptide (g) (FIG. 1 row (g)) to further stabilize the alpha-helical actin-recognition motif of the peptide.

### Example 2

### Labeling of Biomarker Target with Stabilized Peptides in Live Cells

To determine if stabilized peptides can be used to detect biomarkers in live cells, stapled peptide (g) (see FIG. 1, row (g)) was used to label actin filaments in mouse myoblast cells (C2C12 cell line, ATCC® CRL-1772™). Initially, cells were seeded at a density of 10,000 cells/well in 96 well plates in DMEM media and incubated overnight at 37° C. Serial dilutions of each peptide were then transferred to the cells, and the cells were incubated at 37° C. for 3 hours. After washing the cells with phosphate-buffered saline (PBS), Hoechst DNA dye was then added to the cells, which were further incubated at room temperature for 30 minutes before being washed again to remove extra dye. Afterwards, confocal microscopy was used to determine the intracellular localization of the fluorescent-tagged stapled peptide.

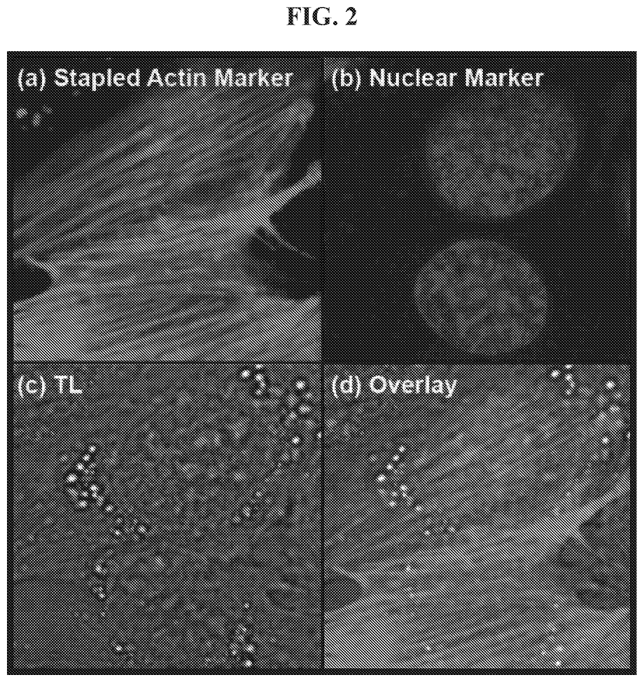

As shown in FIG. 2, stapled peptide (g) entered live cells and selectively bound to and labeled actin filaments (10 μM cellular treatment, room temperature, 2 hours followed by incubation with 50 μM chloroquine for 30 minutes to achieve complete endosomal release and imaged by confocal microscopy). FIG. 2D shows an overlay of three microscopy channels for: (a) the stapled peptide marker for actin in living cells, (b) the Hoechst-labeled nuclei of the cells, and (c) a transmitted light image of the cells. These results show that stabilized peptides can be used to detect biomarkers in live cells effectively. This technology can be applied for, e.g., to FACS and other fluorescent high-throughput screening methods to for example detect overexpression of biomarkers in fixed and living cells.

### Example 3

### Labeling of Biomarker Target with Stabilized Peptides in Fixed Cells

Stapled peptide (g) was also used to detect actin filaments in fixed C2C12 cells. Cells were seeded in 96 well plates at a density of 10,000 cells/well in DMEM media and incubated overnight at 37° C. The cells were fixed using 4% paraformaldehyde in PBS for 20 minutes and then permeabilized using 0.3% Triton-X100 in PBS for 10 minutes. This was followed by the addition of Hoechst DNA dye to the cells to stain nuclei, and FITC-phalloidin (Acti-stain™ 488 phalloidin, Cat. # PHDG1-A, Cytoskeleton, Inc.) to label F actin. After rinsing the cells with PBS, serial dilutions of each peptide were transferred to the cells, and the cells were incubated at 37° C. for 30 minutes. Confocal microscopy was then used to detect the cellular localization of the stapled peptide (g) fused to a fluorescent tag.

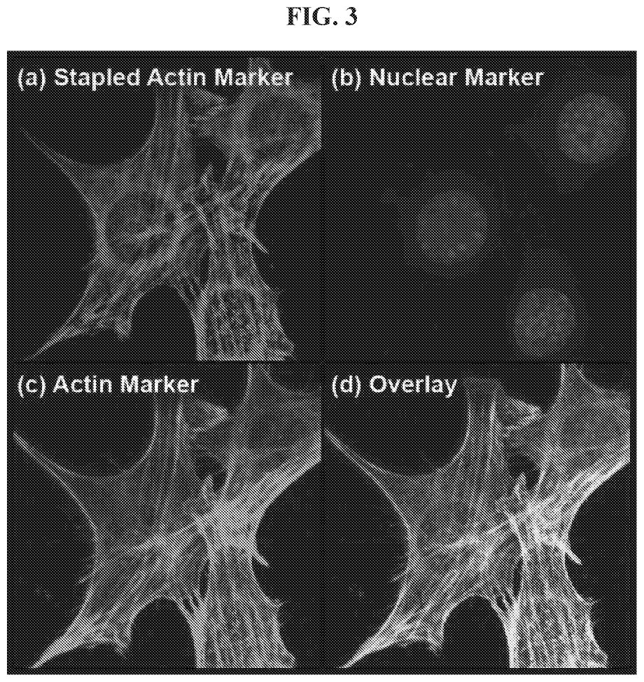

As shown in FIG. 3A, stapled peptide (g) selectively bound to and labeled actin filaments. This peptide co-localized with the actin filament network, which was specifically labeled by FITC-phalloidin, an established marker for actin in fixed cells (compare FIG. 3A and FIG. 3C). FIG. 3D shows an overlay of three microscopy channels for: (a) the stapled peptide marker for actin in fixed cells, (b) the Hoechst-labeled nuclei of the cells, and (c) the actin filaments labeled with FITC-phalloidin. These results show that stabilized peptides can be used to detect biomarkers in fixed cells effectively.

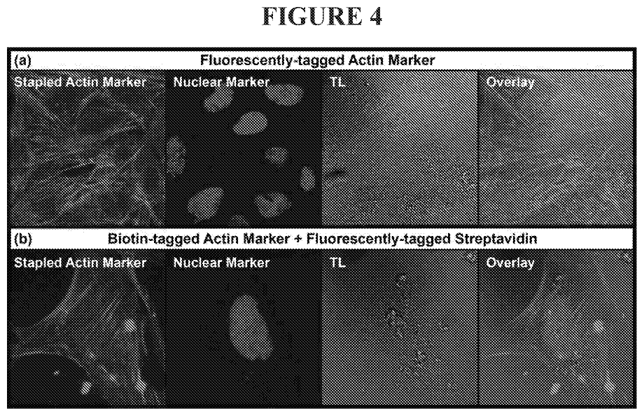

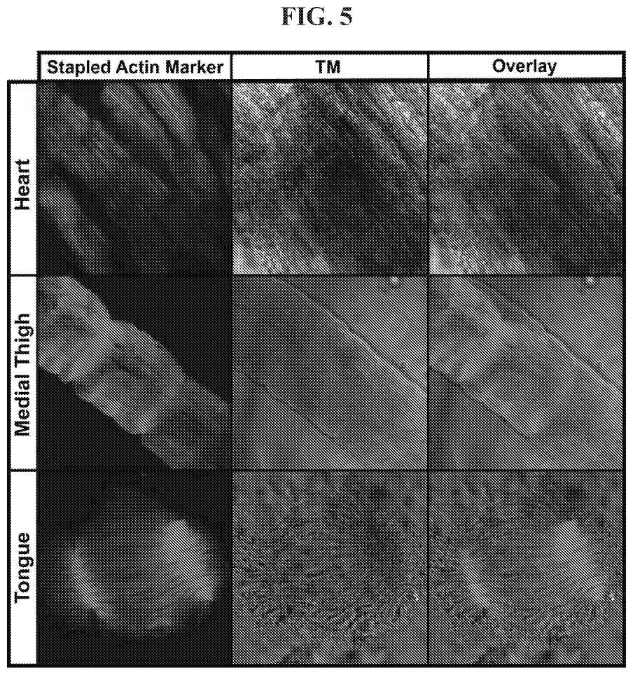

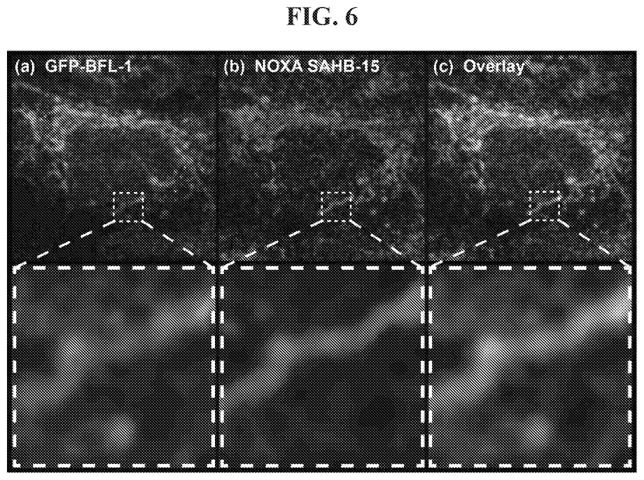

To further demonstrate the versatility of the biomarker detection approach described herein, TAMRA- and biotin-labeled stapled actin-binding peptides were generated for visualization of actin in U2OS cells. In both cases, using direct fluorescence detection of TAMRA-labeled peptide (FIG. 4A) or indirect detection by applying fluorescently-labeled streptavidin to cells treated with the biotinylated construct (FIG. 4B), robust actin detection was observed. The TAMRA-labeled stapled actin-binding peptide was then applied to a series of mouse tissues and demonstrated robust actin visualization in heart, lower extremity, and tongue specimens (FIG. 5).

### Example 4

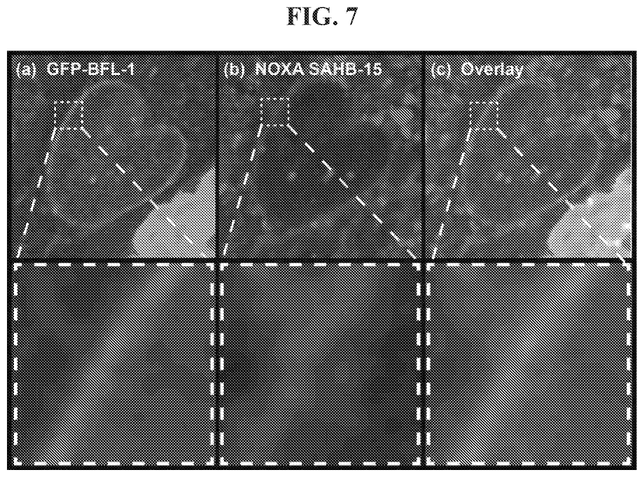

### Development of a Selective Detection Reagent for the Anti-apoptotic Protein BFL-1/A1

Whereas actin was used as an ideal positive control for stapled peptide biomarker development (given the availability of numerous selective actin-detection reagents), developing robust and selective antibodies for the anti-apoptotic BCL-2 family protein BFL-1/A1, for example, has been especially difficult—and exemplary of the general challenges associated with antibody development for various biologically- and therapeutically-relevant targets. Thus, the BFL-1 selective stapled peptide, NOXA SAHB-15 (Guerra et al., Cell Reports, 2018; SEQ ID NO:17) was adapted and applied as a novel method for detecting BFL-1/A1 in live and fixed tissues. Briefly, a C-terminal Lys(Biotin) was added to the NOXA SAHB-15 stabilized peptide of SEQ ID NO:17 (see Guerra et al., Cell Reports, 2018). Given the lack of reliable positive control antibodies for BFL-1/A1, the construct and approach were validated in cells transfected with GFP-labeled BFL-1/A1 and colocalization with the BFL-1/A1-selective stapled peptide at discrete sites in cells was assessed. The colocalization of the stapled peptide with (1) GFP-BFL-1/A1 at its natural distribution, including the mitochondria (FIG. 6), and (2) the nuclear lamina in cells where GFP-BFL-1/A1 was specifically targeted to this discrete, non-endogenous location by GBP-LaminB1, which binds both the nuclear lamina and GFP (FIG. 7), was successfully demonstrated. These data demonstrate the capacity of a stapled peptide marker to selectively engage and visualize its protein target in cells.

### Example 5

### Development of a Detection Reagent for the p53-inhibitory Protein HDM2

In this example, a stapled p53 peptide that targets HDM2 was labeled with biotin as described above (see, e.g., FIG. 1 and Examples 3 and 4) to detect HDM2 in cells and tissues. The construct and approach was validated in cells transfected with GFP-labeled HDM2 and assessed for colocalization with the HDM2-targeting, biotin-labeled stapled peptide, ATSP-7041 (SEQ ID NO:26). Colocalization was successfully demonstrated for the stapled peptide at the nuclear lamina in cells where GFP-HDM2 was specifically targeted to this discrete, non-endogenous location by GBP-LaminB1, which binds both the nuclear lamina and GFP (FIGS. 8A-C). These data demonstrate another example of the capacity of a stapled peptide marker to engage and visualize its protein target in cells, highlighting the broad applicability of this approach.

### Example 6

### Labeling of a Biomarker Target with Stabilized Peptides in a Live Organism.

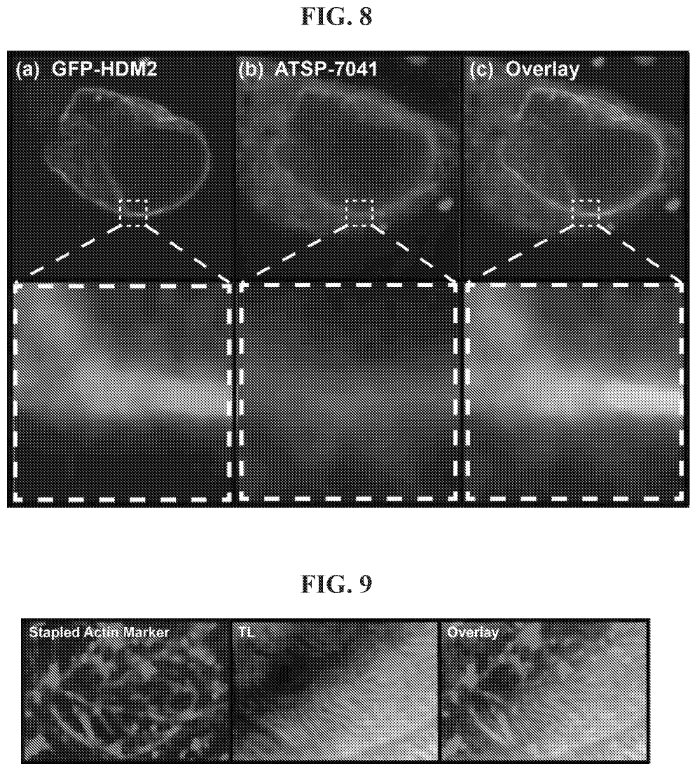

In a further application, a lead TAMRA-labeled stapled actin-binding peptide was incubated with live zebrafish (10 μM, room temperature, 2 hours), followed by treatment with 50 μM chloroquine for 30 minutes to achieve complete endosomal release and was immediately imaged by confocal microscopy (FIG. 9). These data demonstrate the capacity of target-specific stapled peptides to label biomarkers in the cells and tissues of living organisms.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for detecting a biomarker on, outside, or within a cell or tissue, the method comprising:
(a) contacting a cell or tissue with a stabilized peptide that binds to the biomarker, wherein the stabilized peptide is linked to a detectable label; and
(b) detecting the detectable label when the stabilized peptide is bound to the biomarker in the cell or tissue;
thereby detecting the biomarker on, outside, or within the cell or tissue.

2. The method of claim 1, wherein the stabilized peptide comprises a portion of a protein template that binds to the biomarker.

3. The method of claim 1, wherein the stabilized peptide is 4 to 100 amino acids in length.

4. The method of claim 1, wherein the stabilized peptide comprises at least one hydrocarbon staple or stitch.

5. The method of claim 1, wherein the stabilized peptide comprises two hydrocarbon staples.

6. The method of claim 5, wherein the hydrocarbon staples are separated by 2, 3, or 6 amino acids.

7. The method of claim 5, wherein the hydrocarbon staples increase the stability and/or biomarker affinity of the stapled peptide in the cell relative to an identical peptide lacking the hydrocarbon staples.

8. The method of claim 2, wherein the stabilized peptide further comprises at least one amino acid substitution relative to the protein template.

9. The method of claim 8, wherein the at least one amino acid substitution increases the stability and/or biomarker affinity of the stabilized peptide in the cell or tissue relative to an identical stapled peptide lacking the at least one amino acid substitution.

10. The method of claim 1, wherein the detectable label is a radioisotope, a fluorescent dye, an enzyme, or a substrate for an enzyme.

11. The method of claim 1, wherein the detectable label is an affinity tag.

12. The method of claim 1, wherein the cell is a live cell.

13. The method of claim 1, wherein the cell has been fixed.

14. The method of claim 1, wherein the cell is a mammalian cell.

15. The method of claim 1, wherein the cell is a non-mammalian cell.

16. The method of claim 1, further comprising the step of determining the level of detectable label in the cell or tissue relative to a control cell or tissue.

17. The method of claim 1, further comprising determining the intracellular location of the detectable label in the cell.

18. A method for detecting a biomarker on, outside, or within a live cell, the method comprising:
(a) providing a peptide that binds to the biomarker;
(b) introducing at least one staple and/or stitch into the peptide to produce a stabilized peptide that binds to the biomarker;
(c) contacting the cell or tissue with the stabilized peptide that binds to the biomarker; and
(d) detecting the stabilized peptide when the stabilized peptide is bound to the biomarker,
thereby detecting the biomarker on or within the cell.

19. The method of claim 18, wherein the stabilized peptide is linked to a detectable label.

20. The method of claim 19, wherein the detectable label is a radioisotope, a fluorescent dye, an enzyme, or a substrate for an enzyme.

21. The method of claim 19, wherein the detectable label is an affinity tag.

22. The method of claim 18, wherein the stabilized peptide comprises a portion of a protein template that binds to the biomarker.

23. The method of claim 18, wherein the stabilized peptide is 4 to 100 amino acids in length.

24. The method of claim 18, wherein the stabilized peptide comprises at least one hydrocarbon staple.

25. The method of claim 18, wherein the stabilized peptide comprises two hydrocarbon staples.

26. The method of claim 25, wherein the hydrocarbon staples are separated by 2, 3, or 6 amino acids.

27. The method of claim 25, wherein the hydrocarbon staples increase the stability and/or biomarker affinity of the stabilized peptide in the cell relative to an identical peptide lacking the hydrocarbon staples.

28. The method of claim 22, wherein the stabilized peptide further comprises at least one amino acid substitution relative to the protein template.

29. The method of claim 28, wherein the at least one amino acid substitution increases the stability and/or biomarker affinity of the stabilized peptide in the cell relative to an identical stapled peptide lacking the at least one amino acid substitution.

30. The method of claim 18, wherein the cell is a mammalian cell.

31. The method of claim 18, wherein the cell is a non-mammalian cell.

32. The method of claim 18, further comprising the step of determining the level of detectable label in the cell or tissue relative to a control cell or tissue.

33. The method of claim 1, further comprising determining the intracellular location of the detectable label in the cell.

34. A kit for detecting a biomarker on, outside, or within a cell or tissue, the kit comprising:
(a) a stabilized peptide that binds to the biomarker, wherein the stabilized peptide is linked to a detectable label; and
(b) instructions for using the stabilized peptide to detect the biomarker on, outside, or within the cell or tissue.

35. The kit of claim 34, further comprising reagents for detecting the detectable label on, outside, or within the cell or tissue.

36. The kit of claim 34, wherein the stabilized peptide comprises a portion of a protein template that binds to the biomarker.

37. The kit of claim 34, wherein the stabilized peptide comprises at least one hydrocarbon staple.

38. The kit of claim 34, wherein the stabilized peptide comprises two hydrocarbon staples.

39. The kit of claim 38, wherein the hydrocarbon staples increase the stability and/or biomarker affinity of the stabilized peptide in the cell or tissue relative to an identical peptide lacking the hydrocarbon staples.

40. The kit of claim 38, wherein the stabilized peptide further comprises at least one amino acid substitution relative to the protein template.

41. The kit of claim 40, wherein the at least one amino acid substitution increases the stability and/or biomarker affinity of the stabilized peptide in the cell or tissue relative to an identical stabilized peptide lacking the at least one amino acid substitution.

42. The kit of claim 34, wherein the detectable label is a radioisotope, a fluorescent dye, or an enzyme, or a substrate for an enzyme.

43. The kit of claim 34, wherein the detectable label is an affinity tag.

44. The kit of claim 34, wherein the kit comprises instructions for detecting the biomarker on, outside, or within a live cell or tissue.

45. The method of claim 34, wherein the kit comprises instructions for detecting the biomarker on, outside, or within a fixed cell or tissue.

46. The kit of claim 34, wherein the cell is a mammalian cell.

47. The kit of claim 34, wherein the cell is a non-mammalian cell.

48. The kit of claim 34, further comprising instructions and reagents for determining the level of detectable label on, outside, or within the cell or tissue relative to a control cell or tissue.

49. The kit of claim 34, further comprising instructions and reagents for determining the intracellular location of the detectable label in the cell or tissue.

50. A method for selecting a stabilized peptide that binds to a biomarker, the method comprising:
providing a peptide that binds to the biomarker;
introducing at least one staple and/or stitch into the peptide to produce a first stabilized peptide that binds to the biomarker;
contacting the cell or tissue with the first stabilized peptide linked to a detectable label;
detecting that the first stabilized peptide binds to the biomarker better and/or for a longer period of time relative to the peptide and/or a second stabilized peptide generated from the peptide, and
selecting the first stabilized peptide for detecting the biomarker.

51. The method of claim 50, wherein the detectable label is a fluorescent dye.

52. The method of claim 50, wherein the detectable label is a radioisotope, an enzyme, or a substrate for an enzyme.

53. The method of claim 50, wherein the detectable label is an affinity tag.

54. The method of claim 50, wherein the stabilized peptide is 4 to 100 amino acids in length.

55. The method of claim 50, wherein the stabilized peptide comprises at least one hydrocarbon staple.

56. The method of claim 50, wherein the stabilized peptide comprises two hydrocarbon staples.

57. The method of claim 56, wherein the hydrocarbon staples are separated by 2, 3, or 6 amino acids.

58. The method of claim 50, wherein the biomarker is a BCL-2 family protein.

59. The method of claim 50, wherein the biomarker is selected from the group consisting of BFL-1, MCL-1, β-catenin, β-amyloid, tau, α-synuclein, TDP-43, Polycomb protein EED, HDM2, HDMX, WT1, MUC1, LMP2, HPV E6, HPV E7, EGFRvIII, HER-2/neu, MAGE A3, p53, NY-ESO-1, PSMA, GD2, CEA, MelanA/MART1, Ras, gp100, Proteinase3 (PR1), bcr-abl, Tyrosinase, Survivin, PSA, hTERT, Sarcoma translocation breakpoints, EphA2, PAP, ML-IAP, AFP, EpCAM, ERG, NA17, PAX3, ALK, Androgen receptor, Cyclin B1, Polysialic acid, MYCN, RhoC, TRP-2, GD3, Fucosyl GM1, Mesothelin, PSCA, MAGE Al, sLe, CYP1B1, PLAC1, GM3, BORIS, Tn, GloboH, ETV6-AML, NY-BR-1, RGSS, SART3, STn, Carbonic anhydrase IX, PAXS, OY-TES1, Sperm protein 17, LCK, HMWMAA, AKAP-4, SSX2, XAGE 1, 7H3, Legumain, Tie 2, Page4, VEGFR2, MAD-CT-1, FAP, PDGFR-β, MAD-CT-2, Fos-related antigen 1, PCNA, GLP1 receptor, RAS proteins, glucokinase, VLCAD, RSV, or HIV.

60. A method of detecting a biomarker in a solution, the method comprising:
(a) providing a solution comprising an biomarker;
(b) contacting the solution with a stabilized peptide that binds to the biomarker, wherein the stabilized peptide is linked to a detectable label;
(c) detecting the detectable label when the stabilized peptide is bound to the biomarker in the solution;
thereby detecting the biomarker in the solution.

61. The method of claim 60, wherein the solution is blood, serum, plasma, urine, mucous, cerebrospinal fluid, a lavage, pleural fluid, vaginal fluid, semen, peritoneal fluid, or a secretion.

62. The method of claim 61, wherein the secretion is sweat or saliva.

63. The method of claim 60, wherein the solution is blood.

64. The method of claim 60, wherein the stabilized peptide is linked to a solid carrier.

65. The method of claim 64, wherein the solid carrier is a magnetic bead.

66. The method of claim 60, wherein the stabilized peptide binds to the biomarker in solution better and/or for a longer period of time relative to the peptide from which the stabilized peptide is derived or relative to a second stabilized peptide also derived from the same peptide.

67. The method of claim 1, wherein the cell or tissue is a biopsy sample.

68. The method of claim 18, wherein the cell is a biopsy sample.

69. The method of claim 67 or 68, wherein the biopsy sample is a cancer biopsy sample.

70. The method of claim 67 or 68, wherein the biopsy is blood, serum, plasma, urine, mucous, cerebrospinal fluid, or a lavage, pleural fluid, vaginal fluid, semen, peritoneal fluid, or a secretion.

71. The method of claim 70, wherein the secretion is sweat or saliva.

72. The method of claim 67 or 68, wherein the biopsy is blood.

73. The kit of claim 34, wherein the cell or tissue is a biopsy sample.

74. The kit of claim 73, wherein the biopsy sample is a cancer biopsy sample.

75. The kit of claim 34, wherein the cell is part of a bodily fluid.

76. The kit of claim 75, wherein the bodily fluid is blood, serum, plasma, urine, mucous, cerebrospinal fluid, a lavage, pleural fluid, vaginal fluid, semen, peritoneal fluid, or a secretion.

77. The kit of claim 76, wherein the secretion is sweat or saliva.


SEQUENCE LISTING (44 sequences)

SEQ ID NO: 1 - protein, 17 aa, Saccharomyces cerevisiae
MGVADLIKKFESISKEE

SEQ ID NO: 2 - protein, 18 aa, Artificial Sequence
  source: /note=“Description of Artificial Sequence Synthetic peptide”
  MOD_RES (1)..(1): Norleucine
  MOD_RES (6)..(6): Any amino acid available for crosslinking
  MOD_RES (10)..(10): Any amino acid available for crosslinking
  MOD_RES (18)..(18): Lys(TAMRA)
XGVADXIKKXESISKEEX

SEQ ID NO: 3 - protein, 18 aa, Artificial Sequence
  source: /note=“Description of Artificial Sequence Synthetic peptide”
  MOD_RES (1)..(1): Norleucine
  MOD_RES (11)..(11): Any amino acid available for crosslinking
  MOD_RES (15)..(15): Any amino acid available for crosslinking
  MOD_RES (18)..(18): Lys(TAMRA)
XGVADLIKKFXSISXEEX

SEQ ID NO: 4 - protein, 18 aa, Artificial Sequence
  source: /note=“Description of Artificial Sequence Synthetic peptide”
  MOD_RES (1)..(1): Norleucine
  MOD_RES (5)..(5): Any amino acid available for crosslinking
  MOD_RES (9)..(9): Any amino acid available for crosslinking
  MOD_RES (11)..(11): Any amino acid available for crosslinking
  MOD_RES (15)..(15): Any amino acid available for crosslinking
  MOD_RES (18)..(18): Lys(TAMRA)
XGVAXLIKXFXSISXEEX

SEQ ID NO: 5 - protein, 18 aa, Artificial Sequence
  source: /note=“Description of Artificial Sequence Synthetic peptide”
  MOD_RES (1)..(1): Norleucine
  MOD_RES (6)..(6): Any amino acid available for crosslinking
  MOD_RES (10)..(10): Any amino acid available for crosslinking
  MOD_RES (18)..(18): Lys(TAMRA)
XGVADXIKKXESISKEEX

SEQ ID NO: 6 - protein, 18 aa, Artificial Sequence
  source: /note=“Description of Artificial Sequence Synthetic peptide”
  MOD_RES (1)..(1): Norleucine
  MOD_RES (12)..(12): Any amino acid available for crosslinking
  MOD_RES (16)..(16): Any amino acid available for crosslinking
  MOD_RES (18)..(18): Lys(TAMRA)
XGVADLIKKFEXISKXEX

SEQ ID NO: 7 - protein, 18 aa, Artificial Sequence
  source: /note=“Description of Artificial Sequence Synthetic peptide”
  MOD_RES (1)..(1): Norleucine
  MOD_RES (12)..(12): Any amino acid available for crosslinking
  MOD_RES (16)..(16): Any amino acid available for crosslinking
  MOD_RES (18)..(18): Lys(TAMRA)
XGVADLIKKFEXIAKXEX

SEQ ID NO: 8 - protein, 21 aa, Artificial Sequence
  source: /note=“Description of Artificial Sequence Synthetic peptide”
  MOD_RES (9)..(9): (S)-2-(4′-pentenyl) alanine
  MOD_RES (13)..(13): (S)-2-(4′-pentenyl) alanine
IWIAQELRXIGDXFNAYYARR

SEQ ID NO: 9 - protein, 20 aa, Artificial Sequence
  source: /note=“Description of Artificial Sequence Synthetic peptide”
  MOD_RES (1)..(1): (R)-1 acryloylpiperidine-3-carboxamide
  MOD_RES (8)..(8): (S)-2-(4′-pentenyl) alanine
  MOD_RES (12)..(12): (S)-2-(4′-pentenyl) alanine
XIAQELRXIGDXFNAYYARR

SEQ ID NO: 10 - protein, 21 aa, Artificial Sequence
  source: /note=“Description of Artificial Sequence Synthetic peptide”
  MOD_RES (12)..(12): (S)-2-(4′-pentenyl) alanine
  MOD_RES (16)..(16): (S)-2-(4′-pentenyl) alanine
  MOD_RES (17)..(17): Norleucine
DIIRNIARHLAXVGDXXDRSI

SEQ ID NO: 11 - protein, 24 aa, Artificial Sequence
  source: /note=“Description of Artificial Sequence Synthetic peptide”
  MOD_RES (12)..(12): (S)-2-(4′-pentenyl) alanine
  MOD_RES (13)..(13): Norleucine
  MOD_RES (16)..(16): (S)-2-(4′-pentenyl) alanine
QWAREIGAQLRXXADXLNAQYERR

SEQ ID NO: 12 - protein, 25 aa, Artificial Sequence
  source: /note=“Description of Artificial Sequence Synthetic peptide”
  MOD_RES (14)..(14): (S)-2-(4′-pentenyl) alanine
  MOD_RES (15)..(15): Norleucine
  MOD_RES (18)..(18): (S)-2-(4′-pentenyl) alanine
NLWAAQRYGRELRXXSDXFVDSFKK

SEQ ID NO: 13 - protein, 25 aa, Artificial Sequence
  source: /note=“Description of Artificial Sequence Synthetic peptide”
  MOD_RES (14)..(14): (S)-2-(4′-pentenyl) alanine
  MOD_RES (15)..(15): Norleucine
  MOD_RES (16)..(16): PhosphoSer
  MOD_RES (18)..(18): (S)-2-(4′-pentenyl) alanine
NLWAAQRYGRELRXXXDXFVDSFKK

SEQ ID NO: 14 - protein, 25 aa, Artificial Sequence
  source: /note=“Description of Artificial Sequence Synthetic peptide”
  MOD_RES (14)..(14): (S)-2-(4′-pentenyl) alanine
  MOD_RES (15)..(15): Norleucine
  MOD_RES (18)..(18): (S)-2-(4′-pentenyl) alanine
NLWAAQRYGRELRXXDDXFVDSFKK

SEQ ID NO: 15 - protein, 25 aa, Artificial Sequence
  source: /note=“Description of Artificial Sequence Synthetic peptide”
  MOD_RES (13)..(13): (S)-2-(4′-pentenyl) alanine
  MOD_RES (17)..(17): (S)-2-(4′-pentenyl) alanine
AELEVECATQLRXFGDXLNFRQKLL

SEQ ID NO: 16 - protein, 23 aa, Artificial Sequence
  source: /note=“Description of Artificial Sequence Synthetic peptide”
  MOD_RES (1)..(1): (R)-1 acryloylpiperidine-3-carboxamide
  MOD_RES (11)..(11): (S)-2-(4′-pentenyl) alanine
  MOD_RES (15)..(15): (S)-2-(4′-pentenyl) alanine
XEVESATQLRXFGDXLNFRQKLL

SEQ ID NO: 17 - protein, 16 aa, Artificial Sequence
  source: /note=“Description of Artificial Sequence Synthetic peptide”
  MOD_RES (1)..(1): (R)-1 acryloylpiperidine-3-carboxamide
  MOD_RES (4)..(4): Any amino acid available for crosslinking
  MOD_RES (11)..(11): (S)-2-(4′-pentenyl) alanine
XATXLRRFGDXLNFRQ

SEQ ID NO: 18 - protein, 16 aa, Artificial Sequence
  source: /note=“Description of Artificial Sequence Synthetic peptide”
  MOD_RES (1)..(1): (R)-1 acryloylpiperidine-3-carboxamide
  MOD_RES (4)..(4): Any amino acid available for crosslinking
  MOD_RES (11)..(11): (S)-2-(4′-pentenyl) alanine
XATXLREFGDXLNFRQ

SEQ ID NO: 19 - protein, 16 aa, Artificial Sequence
  source: /note=“Description of Artificial Sequence Synthetic peptide”
  MOD_RES (1)..(1): (R)-1 acryloylpiperidine-3-carboxamide
  MOD_RES (4)..(4): Any amino acid available for crosslinking
  MOD_RES (11)..(11): (S)-2-(4′-pentenyl) alanine
XATXLRRAGDXLNFRQ

SEQ ID NO: 20 - protein, 22 aa, Artificial Sequence
  source: /note=“Description of Artificial Sequence Synthetic peptide”
  MOD_RES (15)..(15): (S)-2-(4′-pentenyl) alanine
  MOD_RES (19)..(19): (S)-2-(4′-pentenyl) alanine
RKALETLRRVGDGVXRNHXTAF

SEQ ID NO: 21 - protein, 22 aa, Artificial Sequence
  source: /note=“Description of Artificial Sequence Synthetic peptide”
  MOD_RES (15)..(15): (S)-2-(4′-pentenyl) alanine
  MOD_RES (19)..(19): (S)-2-(4′-pentenyl) alanine
RKALETLRRVGDGAXRNHXTAF

SEQ ID NO: 22 - protein, 22 aa, Artificial Sequence
  source: /note=“Description of Artificial Sequence Synthetic peptide”
  MOD_RES (15)..(15): (S)-2-(4′-pentenyl) alanine
  MOD_RES (19)..(19): (S)-2-(4′-pentenyl) alanine
RKALETLRRVGAGVXRNHXTAF

SEQ ID NO: 23 - protein, 21 aa, Artificial Sequence
  source: /note=“Description of Artificial Sequence Synthetic peptide”
  MOD_RES (3)..(3): Norleucine
  MOD_RES (4)..(4): (S)-2-(4′-pentenyl) alanine
  MOD_RES (8)..(8): (S)-2-(4′-pentenyl) alanine
IWXXQSLXRLGDEINAYYARR

SEQ ID NO: 24 - protein, 20 aa, Artificial Sequence
  source: /note=“Description of Artificial Sequence Synthetic peptide”
  MOD_RES (3)..(3): Norleucine
  MOD_RES (4)..(4): (S)-2-(4′-pentenyl) alanine
  MOD_RES (8)..(8): (S)-2-(4′-pentenyl) alanine
IWXXQELXRLGDEINARYAR

SEQ ID NO: 25 - protein, 16 aa, Artificial Sequence
  source: /note=“Description of Artificial Sequence Synthetic peptide”
  MOD_RES (7)..(7): (S)-2-(4′-pentenyl) alanine
  MOD_RES (14)..(14): (S)-2-(4′-pentenyl) alanine
QSQQTFXNLWRLLXQN

SEQ ID NO: 26 - protein, 14 aa, Artificial Sequence
  source: /note=“Description of Artificial Sequence Synthetic peptide”
  MOD_RES (4)..(4): R-octenyl alanine
  MOD_RES (10)..(10): Cyclobutylalanine
  MOD_RES (11)..(11): (S)-2-(4′-pentenyl) alanine
LTFXEYWAQXXSAA

SEQ ID NO: 27 - protein, 30 aa, Artificial Sequence
  source: /note=“Description of Artificial Sequence Synthetic polypeptide”
  MOD_RES (9)..(9): (S)-2-(4′-pentenyl) alanine
  MOD_RES (13)..(13): (S)-2-(4′-pentenyl) alanine
SNLFSSNRXKILXRTEILNQEWKQRRIQPV

SEQ ID NO: 28 - protein, 27 aa, Artificial Sequence
  source: /note=“Description of Artificial Sequence Synthetic peptide”
  MOD_RES (6)..(6): (S)-2-(4′-pentenyl) alanine
  MOD_RES (10)..(10): (S)-2-(4′-pentenyl) alanine
FSSNRXKILXRTEILNQEWKQRRIQPV

SEQ ID NO: 29 - protein, 30 aa, Artificial Sequence
  source: /note=“Description of Artificial Sequence Synthetic polypeptide”
  MOD_RES (16)..(16): (S)-2-(4′-pentenyl) alanine
  MOD_RES (20)..(20): (S)-2-(4′-pentenyl) alanine
SNLFSSNRQKILERTXILNXEWKQRRIQPV

SEQ ID NO: 30 - protein, 27 aa, Artificial Sequence
  source: /note=“Description of Artificial Sequence Synthetic peptide”
  MOD_RES (13)..(13): (S)-2-(4′-pentenyl) alanine
  MOD_RES (17)..(17): (S)-2-(4′-pentenyl) alanine
FSSNRQKILERTXILNXEWKQRRIQPV

SEQ ID NO: 31 - protein, 30 aa, Artificial Sequence
  source: /note=“Description of Artificial Sequence Synthetic polypeptide”
  MOD_RES (9)..(9): (S)-2-(4′-pentenyl) alanine
  MOD_RES (13)..(13): (S)-2-(4′-pentenyl) alanine
  MOD_RES (16)..(16): (S)-2-(4′-pentenyl) alanine
  MOD_RES (20)..(20): (S)-2-(4′-pentenyl) alanine
SNLFSSNRXKILXRTXILNXEWKQRRIQPV

SEQ ID NO: 32 - protein, 27 aa, Artificial Sequence
  source: /note=“Description of Artificial Sequence Synthetic peptide”
  MOD_RES (6)..(6): (S)-2-(4′-pentenyl) alanine
  MOD_RES (10)..(10): (S)-2-(4′-pentenyl) alanine
  MOD_RES (13)..(13): (S)-2-(4′-pentenyl) alanine
  MOD_RES (17)..(17): (S)-2-(4′-pentenyl) alanine
FSSNRXKILXRTXILNXEWKQRRIQPV

SEQ ID NO: 33 - protein, 24 aa, Artificial Sequence
  source: /note=“Description of Artificial Sequence Synthetic peptide”
  MOD_RES (14)..(14): (S)-2-(4′-pentenyl) alanine
  MOD_RES (18)..(18): (S)-2-(4′-pentenyl) alanine
  MOD_RES (22)..(22): Norleucine
LSQEQLEHRERSLXTLRXIQRXLF

SEQ ID NO: 34 - protein, 35 aa, Artificial Sequence
  source: /note=“Description of Artificial Sequence Synthetic polypeptide”
  MOD_RES (3)..(3): R-octenyl alanine
  MOD_RES (10)..(10): (S)-2-(4′-pentenyl) alanine
  MOD_RES (22)..(22): R-octenyl alanine
  MOD_RES (29)..(29): (S)-2-(4′-pentenyl) alanine
FDXSISQVNXKINQSLAFIRKXDELLHNXNAGKST

SEQ ID NO: 35 - protein, 37 aa, Artificial Sequence
  source: /note=“Description of Artificial Sequence Synthetic polypeptide”
  MOD_RES (1)..(1): Norleucine
  MOD_RES (4)..(4): (S)-2-(4′-pentenyl) alanine
  MOD_RES (8)..(8): (S)-2-(4′-pentenyl) alanine
  MOD_RES (28)..(28): (S)-2-(4′-pentenyl) alanine
  MOD_RES (32)..(32): (S)-2-(4′-pentenyl) alanine
XTWXEWDXEINNYTSLIHSLIEESQNQXEKNXQELLE

SEQ ID NO: 36 - protein, 39 aa, Artificial Sequence
  source: /note=“Description of Artificial Sequence Synthetic polypeptide”
  MOD_RES (10)..(10): (S)-2-(4′-pentenyl) alanine
  MOD_RES (14)..(14): (S)-2-(4′-pentenyl) alanine
  MOD_RES (24)..(24): (S)-2-(4′-pentenyl) alanine
  MOD_RES (28)..(28): (S)-2-(4′-pentenyl) alanine
HGEGTFTSDXSKQXEEEAVRLFIXWLKXGGPSSGAPPPS

SEQ ID NO: 37 - protein, 20 aa, Artificial Sequence
  source: /note=“Description of Artificial Sequence Synthetic peptide”
  MOD_RES (4)..(4): Norleucine
  MOD_RES (13)..(13): (S)-2-(4′-pentenyl) alanine
  MOD_RES (17)..(17): (S)-2-(4′-pentenyl) alanine
EIVXKYIHYKLSXRGYXWDA

SEQ ID NO: 38 - protein, 21 aa, Artificial Sequence
  source: /note=“Description of Artificial Sequence Synthetic peptide”
  MOD_RES (14)..(14): (S)-2-(4′-pentenyl) alanine
  MOD_RES (18)..(18): (S)-2-(4′-pentenyl) alanine
RELVVDFLSYKLSXKGYXWSQ

SEQ ID NO: 39 - protein, 20 aa, Artificial Sequence
  source: /note=“Description of Artificial Sequence Synthetic peptide”
  MOD_RES (13)..(13): (S)-2-(4′-pentenyl) alanine
  MOD_RES (17)..(17): (S)-2-(4′-pentenyl) alanine
  MOD_RES (18)..(18): Norleucine
ALVADFVGYKLRXKGYXXGA

SEQ ID NO: 40 - protein, 18 aa, Artificial Sequence
  source: /note=“Description of Artificial Sequence Synthetic peptide”
  MOD_RES (7)..(7): (S)-2-(4′-pentenyl) alanine
  MOD_RES (11)..(11): (S)-2-(4′-pentenyl) alanine
RRFFGIXLTNXLKTEEGN

SEQ ID NO: 41 - protein, 22 aa, Artificial Sequence
  source: /note=“Description of Artificial Sequence Synthetic peptide”
  MOD_RES (11)..(11): (S)-2-(4′-pentenyl) alanine
  MOD_RES (15)..(15): (S)-2-(4′-pentenyl) alanine
LEVESATQLRXFGDXLNFRQKL

SEQ ID NO: 42 - protein, 27 aa, Artificial Sequence
  source: /note=“Description of Artificial Sequence Synthetic peptide”
  MOD_RES (6)..(6): (S)-2-(4′-pentenyl) alanine
  MOD_RES (10)..(10): (S)-2-(4′-pentenyl) alanine
FSSNRXKILXRTQILNQEWKQRRIQPV

SEQ ID NO: 43 - protein, 21 aa, Artificial Sequence
  source: /note=“Description of Artificial Sequence Synthetic peptide”
  MOD_RES (9)..(9): (S)-2-(4′-pentenyl) alanine
  MOD_RES (13)..(13): (S)-2-(4′-pentenyl) alanine
  MOD_RES (19)..(19): Norleucine
QLTAARLKXLGDXLHQRTXWR

SEQ ID NO: 44 - protein, 18 aa, Artificial Sequence
  source: /note=“Description of Artificial Sequence Synthetic peptide”
  MOD_RES (1)..(1): Norleucine
  MOD_RES (12)..(12): Any amino acid available for crosslinking
  MOD_RES (16)..(16): Any amino acid available for crosslinking
  MOD_RES (18)..(18): Lys(Biotin)
XGVADLIKKFEXIAKXEX